(19) United States
(12) Patent Application Publication
(10) Pub. No.: US 2019/0281936 A1
(43) Pub. Date: Sep. 19, 2019

(54) CONTACT LENS CONTAINER WITH BIOMARKER DETECTION

(71) Applicant: Menicon Co. Ltd., Nagoya (JP)

(72) Inventors: Mouad Lamrani, Geneva (CH); Stephen D. Newman, Singapore (SG)

(21) Appl. No.: 16/351,344

(22) Filed: Mar. 12, 2019

Related U.S. Application Data
(60) Provisional application No. 62/642,897, filed on Mar. 14, 2018.

(51) Int. Cl. A45C 11/00; A61B 5/145; A61L 12/08; G02C 7/04

(52) U.S. Cl. CPC A45C 11/005; A61B 5/14532; A61L 12/086; A61B 5/1455; A61B 5/14546; A61B 5/14507; G02C 7/04

(57) ABSTRACT

A contact lens container can include a body. The body can include a floor, at least one wall connected to the floor, a cavity defined, at least in part, by the floor and the at least one wall, and a sensor oriented to take a measurement of a contact lens solution within the cavity when the cavity is at least partially filled with a contact lens solution.

### CROSS-REFERENCE TO RELATED APPLICATIONS

This application claims priority to U.S. Provisional Patent Application No. 62/642,897 filed on Mar. 14, 2018, the disclosure of which is incorporated herein, in its entirety, by this reference.

### BACKGROUND

When a user wears a contact lens, proteins, lipids, antibodies, and other types of biological materials from the user's tear fluid can be bonded to, adsorbed by, or deposited on the contact lens. In some cases, the bonding of proteins is a result of the protein denaturing, but in other situations, the protein has not denatured before adsorbing to the contact lens. Protein deposits that are visible to the naked eye are most often a result of denaturation. These proteins can build up on the surface of a contact lens, forming protein deposits that impact the transparency of the lens and the integrity of the lens surface. In some cases, the protein deposits trigger an immune reaction and the body produces antibodies in response. These antibodies can cause inflammation, irritation, redness, and itching in the eye.

The build-up of certain biological materials can be indicative of a health condition of the contact lens wearer. Contact lenses have been modified to collect biological material within tear fluid or otherwise bind biological material to the surface of the contact lens to assess a health condition of the wearer.

Traditional approaches for assessing patient health via tear fluid discuss structurally modifying a contact lens to collect information about the composition of the user's tears. U.S. Patent Publication No. 2014/0088381 issued to James Etzkorn, et al. teaches an apparatus, systems, and methods that employ contact lenses to facilitate testing for an analyte present within tear fluid. According to Etzkorn, a contact lens can include a substrate that forms at least part of a body of the contact lens, and one or more cavities disposed within the substrate is configured to collect and store tear fluid over time when the contact lens is worn over an eye. Etzkorn also discloses a contact lens that includes a substrate that forms at least part of a body of the contact lens and one or more receptors disposed on or within the substrate, the one or more receptors being configured to bind to a known ligand.

In another reference, U.S. Patent Publication No. 2004/0181172, issued to Fiona Patricia Carney, et al., a contact lens is disclosed which can be used to collect one or more analytes of interest in a tear fluid from an individual, and in turn, determine the physiological state or health of the individual. Further, this reference teaches that a contact lens for collecting an analyte can be modified to have surface charges present in a density sufficient to impart to the contact lens an increased adsorption of the analyte of interest, a coating including a receptor which specifically binds the analyte of interest, molecular imprints for the analyte of interest, and a core material that is prepared from a composition containing a receptor which binds specifically the analyte of interest.

Further, U.S. Pat. No. 7,429,465 issued to Achim Müller, et al. teaches a process for analyzing an analyte in a hydrogel contact lens following its wear on the eye. The method includes physically or chemically inducing a volume reduction of the hydrogel contact lens, and thereby squeezing the analyte out of the polymer material making up the contact lens and feeding the analyte obtained according to step (a) into an analyzer.

U.S. Pat. No. 6,060,256 issued to Dennis S. Everhart, et al. teaches an inexpensive and sensitive device and method for detecting and quantifying analytes present in a medium. The device includes a metalized film upon which is printed a specific, predetermined pattern of analyte-specific receptors. Upon attachment of a target analyte to select areas of the plastic film upon which the receptor is printed, diffraction of transmitted and/or reflected light occurs via the physical dimensions and defined, precise placement of the analyte. A diffraction image is produced which can be seen with the eye or, optionally, with a sensing device.

U.S. Patent Publication No. 2001/0034500 issued to Wayne Front March, et al. teaches an ophthalmic lens including a receptor moiety that can be used to determine the amount of an analyte in an ocular fluid. The receptor moiety can bind either a specific analyte or a detectably labeled competitor moiety. The amount of detectably labeled competitor moiety which is displaced from the receptor moiety by the analyte is measured and provides a means of determining analyte concentration in an ocular fluid, such as tears, aqueous humor, or interstitial fluid. The concentration of the analyte in the ocular fluid, in turn, indicates the concentration of the analyte in a fluid or tissue sample of the body, such as blood or intracellular fluid. Each of these references are incorporated by reference for all that they contain.

### SUMMARY

In one embodiment, a contact lens container can include a body portion. The body portion can include a floor, at least one wall connected to the floor, a cavity defined, at least in part, by the floor and the at least one wall, and a sensor oriented to take a measurement of a contact lens solution within the cavity when the cavity is at least partially filled with a contact lens solution.

The sensor can be incorporated into the body portion. The sensor can include at least portion that is exposed within the cavity. The sensor can include at least one energy source. The at least one energy source can include a light transmitter. The light transmitter can include at least one mirror. The light transmitter can include at least one optical window. The light transmitter can include at least one diffraction grating. The light transmitter can include at least one light source. The at least one energy source can be an acoustic source. The at least one energy source can be a radiation source. The at least one energy source can be exposed within the cavity. The sensor can be a receiver oriented to detect a characteristic of energy from the at least one energy source from within the solution. The sensor can be located on a far side of the cavity from the at least one energy source. The sensor can be located on a same side of the cavity as the at least one energy source. The contact lens container can include a lid portion shaped to interlock with the body portion. The sensor can be incorporated into the lid portion.

The contact lens container can include a user interface in communication with the at least one sensor. The user interface can be incorporated into the body portion. The contact lens container can include a lid portion shaped to interlock with the body portion, and the user interface can be incorporated into the lid portion. The user interface can be in wireless communication with the at least one sensor. The user interface can be configured to present an option to initiate an analysis of the contact lens solution. The user interface can be configured to present an option to schedule an analysis of the contact lens solution. The user interface can be configured to present an option to send measurements of the analysis to a remote device. The user interface can be configured to present a result of the analysis. The sensor can be an optical spectrometer. The sensor can include configured to take separate measurements at different energy intensities. Taking separate measurements at different energy intensities can include taking measurements at different optical wavelengths.

The sensor can include a near infrared spectrometer configured to provide light in the cavity in a wavelength region of approximately 900 nm to approximately 2500 nm. The near infrared spectrometer can include a light source directed substantially perpendicular to a contact lens when a contact lens is deposited into the cavity. The near infrared spectrometer can be configured to provide the light in a wavelength of approximately 1300 nm to approximately 1600 nm. The sensor can be configured to emit and absorb the light in response to a user request. The sensor can be in wireless communication with an external electronic device, and the user request is received from the electronic device. The sensor can be configured to repeatedly emit and absorb the light automatically in predetermined intervals. The predetermined intervals include every hour after a contact lens is deposited into the cavity until the contact lens is removed from the cavity.

The sensor can include a microfluidic disposable strip secured to the body portion, the microfluidic disposable strip can be configured to collect a portion of the contact lens solution and one or more solutes dissolved in the contact solution from a contact lens placed in the cavity with the contact lens solution. The microfluidic disposable strip can include a substrate having an immuno-based platform configured to change colors responsive to one or more biomarkers collecting on the microfluidic disposable strip. The sensor also can include an ultraviolet spectrometer configured to quantitatively analyze a color change of the substrate on the microfluidic disposable strip. The microfluidic disposable strip can include a substrate having a fluorescent immuno-based platform configured to change colors in response to one or more biomarkers collecting on the microfluidic disposable strip. The sensor can include a fluorescent reader unit configured to quantitatively analyze a color change of the substrate on the microfluidic disposable strip. The one or more biomarkers can be associated with ocular surface inflammation and can include at least one of a cytokine, an enzyme, an immunoglobulin, a protein, a peptide, or a lipid.

The contact lens container also can include a microfluidic disposable strip secured to the body portion. The microfluidic disposable strip can be configured to collect a portion of the contact lens solution and one or more solutes dissolved in the contact solution from a contact lens placed in the cavity with the contact lens solution. The microfluidic disposable strip can include a substrate having an immuno-based platform configured to change colors responsive to one or more biomarkers collecting on the microfluidic disposable strip. The light transmitter can include an ultraviolet spectrometer configured to quantitatively analyze a color change of the substrate on the microfluidic disposable strip. The microfluidic disposable strip can include a substrate having a fluorescent immuno-based platform configured to change colors responsive to one or more biomarkers collecting on the microfluidic disposable strip. The light transmitter can include a fluorescent reader unit configured to quantitatively analyze a color change of the substrate on the microfluidic disposable strip. The one or more biomarkers can be associated with ocular surface inflammation and include at least one of a cytokine, an enzyme, an immunoglobulin, a protein, a peptide, or a lipid.

In one embodiment, a contact lens container includes a body portion. The body portion can include a floor, at least one wall connected to the floor, a cavity defined, at least in part, by the floor and the at least one wall, and a light transmitter incorporated into the container and oriented to transmit a light into the cavity.

The contact lens container can include a lid portion shaped to interlock with the body portion. The light transmitter can be incorporated into the lid portion. The light transmitter can be incorporated into the body portion. The light transmitter can operate within a range that includes at least a portion of a visible light range. The light transmitter can operate within a range that includes at least a portion of an infrared light range. The light transmitter can operate within a range that includes at least a portion of an ultraviolet light range. The contact lens container can include a light receiver positioned proximate to the cavity to receive at least a portion of light from the light transmitter. The contact lens container can include a light receiver positioned within the cavity to receive at least a portion of light from the light transmitter.

In one embodiment, a contact lens container can include a body portion. The body portion can include a floor, at least one wall connected to the floor, a cavity defined, at least in part, by the floor and the at least one wall, and a light transmitter incorporated into the container and oriented to transmit a light within the cavity.

In one embodiment, ascertaining a health condition of a user includes sending information about biomarkers from a contact lens container to a computing device.

The information can be obtained by measuring at least an optical transmittance through the contact lens solution. The optical transmittance can be obtained by measuring at least a first optical transmittance at a first wavelength through the contact lens solution and a second optical transmittance at a second wavelength through the contact lens solution. The contact lens container can include a light source and a diffraction grating positioned to diffract light from the light source. The contact lens container can include a tilt mechanism attached to the diffraction grating. The contact lens can include a slit positioned to receive at least some of the light from the light source after being diffracted with the diffraction grating. In some embodiments, the tilt mechanism is configured to different a range of wavelengths through the slit by positioning the diffraction grating.

The method can include transmitting a first wavelength of light through a contact lens solution within the contact lens container, obtaining a first optical transmittance measurement of the first wavelength of light through the contact lens solution, transmitting a second wavelength of light through a contact lens solution by moving the diffraction grating with the tilt mechanism, and obtaining a second optical transmittance measurement of the second wavelength through the contact lens solution.

The method can include analyzing a microfluidic disposable strip in the contact solution to obtain the information. The microfluidic disposable strip can be analyzed with an ultraviolet spectrometer. The microfluidic disposable strip can be analyzed with a fluorescent reader unit.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates an exemplary cross-sectional view of a contact lens positioned on an eye, in accordance with the present disclosure.

FIG. 2 illustrates an exemplary cross-sectional view of biomarkers adhered to a contact lens, in accordance with the present disclosure.

FIG. 3 illustrates a cross-sectional view of an example contact lens in solution, in accordance with the present disclosure.

FIG. 4 illustrates a cross-sectional view of an example of running a test on the solution containing biomarkers from a contact lens, in accordance with the present disclosure.

FIG. 5 illustrates a cross-sectional view of an example of a light transmitter, in accordance with the present disclosure.

FIG. 6 illustrates a cross-sectional view of an example of a light transmitter, in accordance with the present disclosure.

FIG. 7 illustrates a block diagram of an example of a health condition system, in accordance with the present disclosure.

FIG. 8 illustrates a block diagram of an example of a database, in accordance with the present disclosure.

FIG. 9 illustrates a schematic view of an example of a health condition system, in accordance with the present disclosure.

FIG. 10 illustrates a block diagram of a method of determining a health condition, in accordance with the present disclosure.

FIG. 11 illustrates a block diagram of a method of determining a health condition, in accordance with the present disclosure.

FIG. 12 illustrates an example of a contact lens storage container, in accordance with the present disclosure.

FIG. 13 illustrates a cutaway view of an example of a contact lens storage container, in accordance with the present disclosure.

FIG. 14 illustrates a cutaway view of an example of a contact lens storage container, in accordance with the present disclosure.

FIG. 15 illustrates a cutaway view of an example of a contact lens storage container, in accordance with the present disclosure.

FIG. 16 illustrates a cutaway view of an example of a contact lens storage container, in accordance with the present disclosure.

FIG. 17 illustrates a perspective view of an example of a contact lens storage container, in accordance with the present disclosure.

FIG. 18-20 illustrate infrared absorbance charts of solution samples at various wavelengths, in accordance with the present disclosure.

FIG. 21 illustrates a calibration curve of glucose, in accordance with the present disclosure.

Throughout the drawings, identical reference numbers designate similar, but not necessarily identical, elements.

### DETAILED DESCRIPTION

A healthy human eye is coated with tear fluid. Generally, the tear fluid includes a base mucous layer that coats the cornea of the eye, an aqueous layer, and a lipid layer that protects the aqueous layer by forming an outer hydrophobic barrier that helps to retain the aqueous layer against the mucous layer. The aqueous layer includes metabolites, proteins, electrolytes, and other constituents. The make-up of the tear fluid can result, in part, from a physiological response to an illness or an allergy. In some cases, the make-up of the tear fluid can represent the physiological expression of an individual's unique DNA.

The present exemplary systems and methods include a method of using the constituents of the tear fluid as a characteristic, including predisposition biomarkers, screening biomarkers, staging biomarkers, prediction biomarkers and prognostic biomarkers, that can be analyzed to determine or predict a health condition of a user or to identify a physiological process, pathological process, or the effect of a therapy. These biomarkers can be collected on a contact lens worn by the user. Any appropriate type of contact lens can be used to collect the biomarkers. According to one exemplary embodiment, unaltered commercially available contact lenses for corrective vision can be used to collect the biomarkers. Biomarkers, such as proteins, generally start to bind to these contact lenses as soon as the contact lenses are placed on the user's eye. Without modifying the commercially available contact lens, the contact lens can bind to these proteins, electrolytes, and/or other biomarkers in the tear fluid.

Generally, a user removes the contact lens after wearing it for a period of time. Often, before the user retires to bed, the user removes the contact lens and places the contact lens in a storage container for the night. The storage container can include a storage solution that disinfects the contact lens and also breaks down the build-up on the contact lens. The storage solution can be an aqueous solution that causes the build-up on the contact lens to dissolve into the solution. According to one exemplary embodiment, a mechanical or fluid agitator can be added to the storage container to aid in the release of the biomarkers. After a period of time, the storage solution can be replaced with fresh storage solution to reduce the concentration of tear fluid constituents in the fluid.

The storage solution can be analyzed to determine the type and/or concentration of biomarkers that entered the storage solution from the contact lens. In some cases, the solution can be analyzed with the contact lens in the solution. In other examples, the contact lens is removed from the solution before analyzing the biomarkers.

Any appropriate type of sensor can be used to identify the type and/or concentration of the biomarkers. In some instances, the sensor is incorporated into the contact lens storage container. In this example, the sensor can be an optical spectral analyzer that passes light through the cavity of the storage container holding the storage solution from a light source to a light receiver. The receiver can measure the amount the light's optical transmittance through the storage solution. In some instances, the spectral analyzer passes light through the storage solution at isolated predetermined wavelengths and measures the optical transmittance at each of the predetermined wavelength ranges. Each of the recorded transmittances can correlate to the presence of specific kinds of biomarkers and their concentrations.

In other examples, the sensor is incorporated into a hand-held device. In one example, the sensor can be incorporated into the user's mobile device, such as a smart phone and/or electronic tablet. In one of these examples, the user can direct a beam of light into the storage solution and measure a reflection or a spectral analysis.

In some examples, the measurements are associated with an amount of time that the user wore the contact lens. For example, the user can interact with a user interface to the sensor to input how long the user wore the contact lenses. In some cases, the user can be requested to input the number of hours that the user wore the contact lens. In other examples, the user can be requested to input the number of days that he or she wore the contact lenses, whether the user removed the contact lenses during the night, the time of when the storage solution was last replaced, other factors that can affect the concentration of biomarkers in the storage solution, or combinations thereof. In some examples, the storage container detects the presence or removal of a lens and determines the user's wear time with the lens based on the time the lens is out of the storage container.

In some examples, the sensor can record the measurements to determine a measurement level of each of the desired biomarkers. In some examples, the sensor can record the measurements in real time. Further, the sensor can include local and/or cloud based logic to determine the type concentration, and/or other characteristics of the varying kinds of biomarkers.

In some cases, the sensor can use learning algorithms, predictive models, data correlation models, clustering models, any other appropriate computational techniques, and combinations thereof. In some cases, the algorithms applied to data collected from the sensor can include support vector machines, neural networks, decision trees, Gaussian mixture models, hidden Markov methods, and wavelet analysis. The models used to learn from data can include but are not limited to, anomaly detection models, clustering models, classification models, regressions models or summarization models. In some examples, the sensor can include a database that stores the correlation between the identification/concentration of the biomarkers and an actual or predicted health condition of the user.

The measurements can be sent to a computing device that processes the information retrieved from the sensor. In some cases, at least some computations are performed by the sensor before sending data to a computing device, where the computations are finished. In other examples, the sensor sends raw data to the computing device. In this example, all data processing, including data cleaning, data management, data mining, and any application specific issues, is performed at a location remote to the sensor. In some examples, information processing can include data preprocessing, for example, in order to format or modify the data for use in subsequent processing. In some examples, data preprocessing can include formatting for matrix computations, data normalization, data synchronization, and data filtering.

The determinations of the type of biomarkers, the characteristics of the biomarkers, such as the concentration of the biomarkers, chemo-metric data such as ratio kinetics, peak, plateau, time constant, decay, and so forth can be compared to data points stored in a database. The database can be local to the computing device or the computing device can have remote access to the database. The data in the database can correlate the different types and concentrations of biomarkers with health conditions, such as eye health conditions, allergic conditions, other physiological conditions, or combinations thereof. In some examples, the data in the database can be used as input or training data to implement supervised machine learning techniques, or other statistical learning approaches to solve prediction inference, or other data mining problems related to health conditions, such as eye health conditions, allergic conditions, other physiological conditions, or combinations thereof.

In some examples, the database is in communication with multiple users and data sources. As data regarding the storage solution of a user is collected, data from each of the users can contribute to the information in the database. In some examples, data collection can automatically launch a data management system of the database. In some examples, the data management system or another process can incorporate additional data into the database, such as the health conditions of each of the users. As result, the correlations in the database can be built from reports from the users. In some examples, patient data can be used as predictors in a statistical machine learning process. In some cases where the database is built using thousands of users, the database's input can identify correlations between health conditions and specific levels of different types of biomarkers that are unknown to the scientific community. Thus, even before scientific studies can be conducted to find a correlation between a biomarker and a health condition, the database can send information related to the diagnosis of a disease, a disease severity assessment, a risk stratification, a therapeutic decision or request, a recommendation to the user to be tested for a specific type of condition, or combinations thereof.

These principles allow a super multivariate database to be built that correlates the health conditions of the users with varying biomarker parameters. For example, the database can include supplementary user data such as age, gender, weight, height, and the like. These principles also allow the user to have a non-invasive procedure to measure the biomarkers. Further, in those cases where the user is already storing and cleaning his or her contact lens from time to time, the user can incurred little to no additional effort to measure the biomarkers and receive reports on at least some of his or her health conditions.

In some cases, the database can include correlations between a user's biomarker profile and other types of biomarker characteristics, to determine a type of contact lens for the user. The biomarkers can indicate that the user has an allergic reaction to a particular type of contact lens, and the database can include information on other types of contact lenses that the user is likely to be non-allergic to. In other examples, the database can indicate that the user has an allergy to, or the contact lens has another property that would suggest that another type of contact lens is better suited for the user, but the database may not provide alternative options to the user. The user, however, is better off for knowing which of the contact lens types do not have a high likelihood of being comfortable for the user, so that the user can avoid purchasing these types of contact lenses. In other examples, the system can look to a different source, other than the database, to find information on the types of contact lenses that are more likely to be well suited for the user.

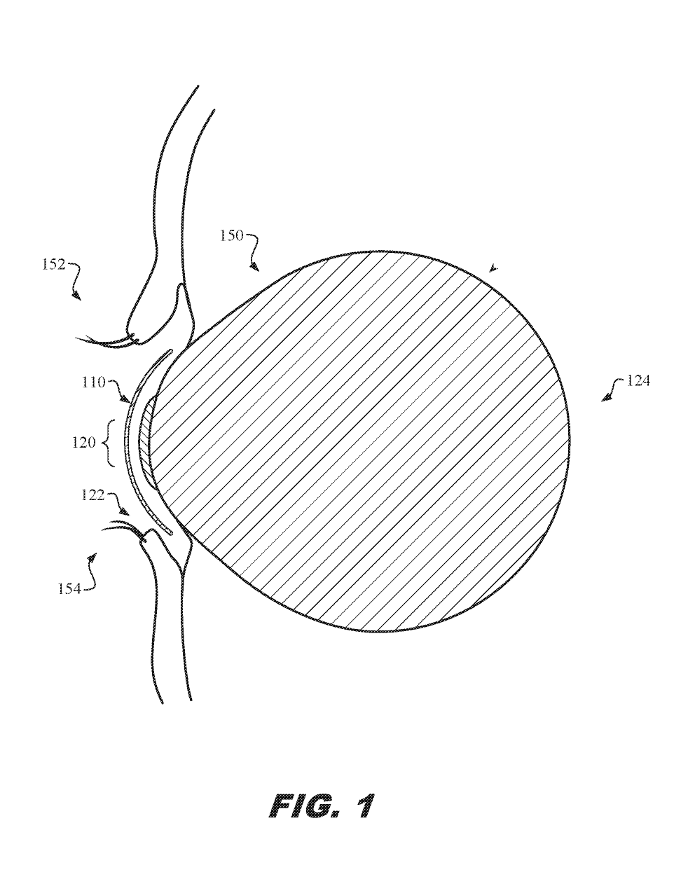

Referring now to the figures, FIG. 1 depicts an example of a contact lens 110 situated on the outside of a human eye 150. The contact lens 110 spans a portion of the outside surface of the exposed portion of the eye 150. An upper portion of the contact lens 110 is adjacent a set of eyelashes 152 of the upper eye-lid. The contact lens 110 can include a posterior side that is in contact with the eye 150, and an anterior side that is opposite the posterior side. As the lid travels over the eye 150, the eye-lid moves across the anterior side of the contact lens 110.

A user can wear the contact lens for vision correction purposes. In this example, the contact lens can include an optic zone 120 and a peripheral zone 122. The optic zone 120 can include a region that focuses light to the center of the user's retina 124. The peripheral zone 122 can contact the eye over the sclera and/or part of the cornea. While this example discloses using commercially available contact lenses configured for vision correction to be worn on the eye, other types of contact lenses can be used in accordance with the principles described in the present disclosure. For example, the contact lens may not include a curvature or features that correct vision.

The contact lens 110 can be a soft contact lens, a rigid gas permeable (RGP) contact lens, an orthokeratology contact lens, another type of contact lens, or combinations thereof. The contact lens can be made of any appropriate type of material. A non-exhaustive list of materials that can be used to construct the contact lens include any appropriate silicone material and/or hydrogel material. Such material can be formed of polymers, such as tefilcon, tetrafilcon A, crofilcon, helfilcon A&B, mafilcon, polymacon, hioxifilcon B, lotrafilcon A, lotrafilcon B, galyfilcon A, senofilcon A, sifilcon A, comfilcon A, enfilcon A, lidofilcon B, surfilcon A, lidofilcon A, alfafilcon A, omafilcon A, vasurfilcon A, hioxifilcon A, hioxifilcon D, nelfilcon A, hilafilcon A, acofilcon A, bufilcon A, deltafilcon A, phemfilcon A, bufilcon A, perfilcon, etafilcon A, focofilcon A, ocufilcon B, ocufilcon C, ocufilcon D ocufilcon E, ocufilcon F, phemfilcon A, methafilcon A, methafilcon B, vilfilcon A, other types of polymers, monomers, or combinations thereof. These materials can include various combinations of monomers, polymers, and other materials to form the material that makes up the contact lens.

In one embodiment, the contact lens material is made of hydrogel polymers without any silicone. This can be desirable to increase the wettability of the contact lens. In another embodiment, the contact lens material is made of silicone hydrogel material.

The tear fluid in the ocular cavity can come into contact with the contact lens. In some examples, the entire surface area of the contact lens comes into contact with the tear fluid. The constituents of the tear fluid can include lipids, electrolytes, metabolites, proteins, antibodies, other types of compounds, or combinations thereof. These constituents can be biomarkers that can be indicative of a health condition of the user. The biomarkers can bind to the contact lens.

A non-exhaustive list of biomarkers from the tear fluid that can be of interest includes, but is not limited to, electrolytes, sodium, potassium, chloride, phenylalanine, uric acid, galactose, glucose, cysteine, homocysteine, calcium, ethanol, acetylcholine and acetylcholine analogs, ornithine, blood urea nitrogen, creatinine, metallic elements, iron, copper, magnesium, polypeptide hormones, thyroid stimulating hormone, growth hormone, insulin, luteinizing hormones, chorionogonadotrophic hormone, obesity markers, leptin, serotonin, medications, dilantin, phenobarbital, propranolol, cocaine, heroin, ketamine, hormones, thyroid hormones, ACTH, estrogen, cortisol, progesterone, histamine, cytokines, lipids, cholesterol, apolipoprotein Ai, proteins and enzymes, lactoferrin, lysozyme, tear-specific prealbumin or lipocalin, osmolarity, matrix metaloproteinase-9, albumin, complement factors, coagulation factors, liver function enzymes, heart damage enzymes, ferritin, virus components, immunoglobulins such as IgM, IgG, IgE, proteases, protease inhibitors, lactate, ketone bodies, other types of biomarkers, or combinations thereof.

In some cases, a commercially available contact lens can have surface properties to allow the biomarkers to bind to the contact lens without any modifications. Conventionally, protein build-ups and other types of build-ups on contact lens are considered a problem on regular contact lens that do not have surface modifications to enhance a biomarker's ability to bind to the contact lens. In other examples, the contact lens can be modified to enhance the binding ability of the biomarkers or just for specific biomarkers. In those examples where the surface of the contact lens can be modified to enhance an ability to bind to the biomarkers, the binding enhancements can be made to any appropriate location on the contact lens, including, but not limited to, the peripheral zone, the optical zone, the anterior side of the contact lens, the posterior side of the contact lens, other areas of the contact lens, or combinations thereof.

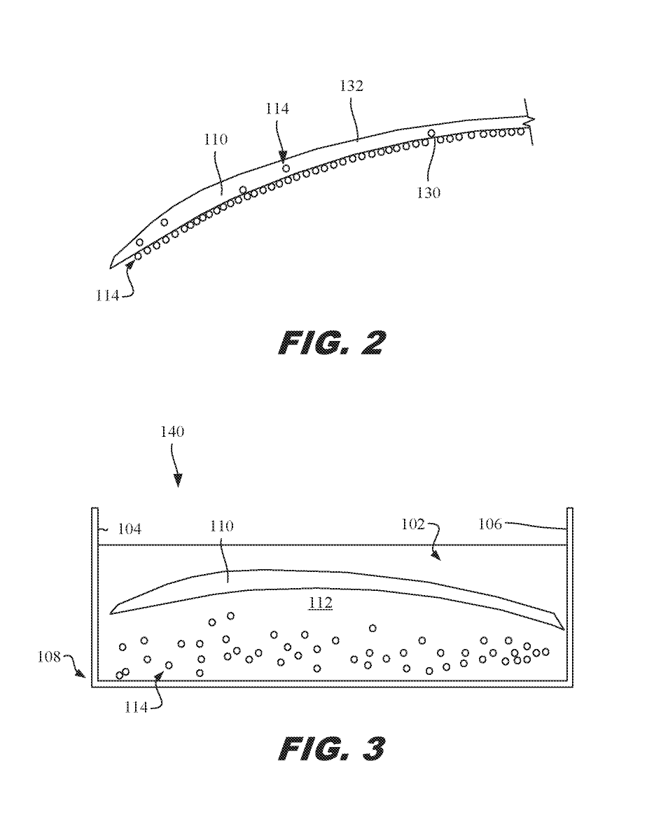

FIG. 2 depicts an example of biomarkers 114 attached to the posterior surface 130 of the contact lens. While this example depicts the biomarkers 114 attached to the posterior surface 130 of the contact lens, the biomarkers 114 can be attached to just the anterior surface 132 or to both the anterior surface 132 and posterior surface 130 of the contact lens 110. In some examples, the biomarkers 114 can be adsorbed, absorbed, bonded, covalently bonded, ionically bonded, adhered, cohered, or otherwise connected to a surface of the contact lens 110. In some examples, the biomarkers 114 are incorporated into the thickness of the contact lens 110. In some examples, the sensor monitors the osmolarity of the contact lens hydrated with tears or other optical fluid.

When the contact lens 110 is removed from the user's eye, the biomarkers 114 can stay with the contact lens 110, as depicted in FIG. 2. The biomarkers 114 that are attached to the contact lens 110 can be related to the amount of time that the contact lens 110 was on the eye. In some examples, the contact lens 110 can be worn by the user during that day and removed at nighttime. Under these circumstances, biomarkers 114 can cover a substantial amount of the contact's lens surface area. However, in other examples, the contact lens 110 can be worn by the user for a shorter period of time. In one specific instance, a patient can be provided with a contact lens 110 for a period of minutes in a doctor's office to collect biomarkers 114 for analysis. In other examples, a patient can be instructed to wear a contact lens 110 for a matter of hours or even longer than a single day, to collect the desired about of biomarkers 114.

FIG. 3 depicts an example of a contact lens 110 in a storage container 140 with an internal cavity 102. The cavity 102 is defined by a first wall 104 and a second wall 106 that are connected together at the walls' ends 108. A contact lens 110 and a solution 112 are also disposed within the cavity.

The solution 112 can include a cleansing agent, such as a hydrogen peroxide or another type of agent to clean the contact lens and kill bacteria, fungus, other types of germs, or combinations thereof. The solution 112 can be an off-the-shelf type of storage solution that hydrates and cleans the contact lens. The storage solution 112 can cause the biomarkers 114 to dissolve into the solution 112, thereby cleaning the contact lens 110. The contact lens 110 stays in the storage solution 112 until the contact lens 110 is later retrieved by the user for wearing. In some examples, the contact lens 110 is immersed into the solution for a short period of time, such as a couple of minutes. In other examples, the contact lens 110 can remain in the solution for multiple hours, such as overnight. With the biomarkers 114 removed from the contact lens 110, the biomarkers 114 are in the solution 112 where the biomarker types and their respective concentrations can be analyzed.

The biomarkers 114 can be removed from the contact lens 110 without adversely affecting the contact lens 110. In those examples, the contact lens 110 can be re-worn by the user. In some cases, the contact lens 110 is removed from the solution 112 so that the contact lens 110 is not affected by the testing mechanism performed on the solution. In other examples, the contact lens 110 remains in the solution 112 while the solution 112 is analyzed, but the analysis does not adversely affect the contact lens 110 so that the contact lens 110 can be re-worn by the user.

In some examples, the biomarkers 114 can be analyzed in the storage container 140. In other examples, the solution 112 can be transferred to another type of device with a sensor for taking the measurements. In yet another example, a hand-held device that includes a sensor can be used to perform the analysis on the solution.

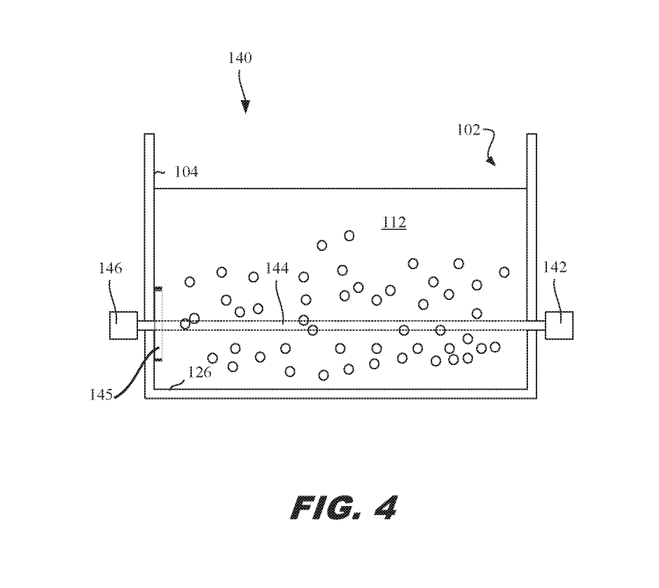

One exemplary approach to analyzing the solution is depicted in FIG. 4. In this example, an optical spectral analyzer is incorporated into the storage container 140. In the example of FIG. 4, a storage container 140 for a contact lens 110 includes a cavity 102 that is defined by at least one wall 104 that is connected by a floor 126. In some cases, a single circular wall defines at least a portion of the cavity 102. In other examples, multiple independent walls are joined together to define the cavity 102.

A light transmitter 142 is incorporated into a first side of the cavity. The light transmitter 142 can transmit any appropriate type of light. In some examples, the light transmitter 142 transmits an incandescent light, a fluorescent light, a halogen light, an infrared light, a visible light, an ultraviolet light, another type of light, or combinations thereof. The light transmitter 142 can include a bulb, a diode, or another source that can be turned on and off with a switch. The light transmitter 142 can include one or multiple light sources. Light sources in the light transmitter 142 can be configured to provide light at within a desired wavelength. For example, the light transmitter 142 can include one or more light sources to transmit light having wavelengths in the ultraviolet region and/or the infrared region.

The light transmitter 142 can be oriented to direct a beam 144 of light through the solution 112 to a light receiver 146 or a detector. As the beam 144 of light is transmitted through the solution 112, a portion of the light is absorbed by the solution, depending on its contents. A solution 112 with a different type of biomarker 114 can have a different light transmittance through the solution 112. Further, a solution 112 with a different concentration of the same biomarker 114 can also exhibit a different light transmittance.

In some examples, the light transmitter 142 can isolate a range of wavelengths to be transmitted independently through the solution 112. The transmittance for each wavelength can be measured. Certain biomarkers in the solution 112 might not, according to some embodiments, affect the optical transmittance at a first wavelength, but can affect the optical transmittance at a second wavelength. Thus, by transmitting light at multiple wavelengths, a more refined measurement of the solution's composition can be measured. The measured transmittances at each wavelength can be compared to other solutions with known types and known amounts of biomarkers. Thus, the measured transmittance levels can be correlated to the types and concentration of the biomarkers 114 in the solution 112.

Other types of spectroscopic methods can be used to identify the types and concentration of the biomarkers in the solution. In some examples, measuring a frequency rather than a wavelength can be performed by the spectral analyzer. A non-exhaustive list of other types of spectroscopic mechanisms for analyzing the solution can include atomic absorption spectroscopy, attenuated total reflectance spectroscopy, electron paramagnetic spectroscopy, electron spectroscopy, Fourier transform spectroscopy, gamma-ray spectroscopy, infrared spectroscopy, laser spectroscopy, mass spectrometry multiplex or frequency-modulated spectroscopy, near-infrared (NIR) spectroscopy, Raman spectroscopy, ultraviolet spectroscopy, and x-ray spectroscopy.

The light receiver 146 or detector can also correspond to the wavelength of light emitted by the light transmitter 142. For example, an infrared light transmitter 142 can correspond to a light receiver configured to detect light at an infrared wavelength. In some embodiments, the light receiver 146 is configured to detect light at multiple spectra, such as both ultraviolet and infrared. The light receiver 146 can be configured to detect one or more of incandescent light, fluorescent light, halogen light, infrared light, visible light, ultraviolet light, another type of light, or combinations thereof.

While the example of FIG. 4 includes the light transmitter 142 and the light receiver 146 on different sides of the cavity walls, the light transmitter 142 and the light receiver 146 can be on the same side of the cavity 102. In such an example, the light transmitter 142 can cause a reflection of the light that was emitted from the light transmitter 142 with the light receiver 146.

In some embodiments, a microfluidic disposable strip 145 also can be used in analyzing a solution 112. The microfluidic disposable strip 145 can be incorporated into or adhered to the body portion of the lens container. For example, in FIG. 4, a microfluidic disposable strip 145 is positioned on the first wall 104. In other embodiments, the microfluidic disposable strip 145 can be positioned on the floor 126, the second wall 106, or elsewhere in the body portion or the lid portion of the lens container. For example, a microfluidic disposable strip 145 can be positioned anywhere on at least one of the body portion or the lid portion such that the microfluidic disposable strip 145 is at least partially immersed in lens solution in the cavity of the lens container. When the cavity is at least partially filled with a contact solution and the contact lens is in the standard position in the lens container, the microfluidic strip sensor 145 can, according to one embodiment, continuously collect contact lens solution and solutes dissolved from worn contact lens.

Sensing of the microfluidic disposable strip can be performed using an immuno-based platform with colorimetric reading. For example, in some embodiments, the light transmitter 142 and the light receiver 146 include an ultraviolet spectrometer incorporated into the body portion of the lens container. The ultraviolet spectrometer can be used to quantitatively analyze a color change of a substrate on the microfluidic disposable strip 145 to target specific antibodies on the microfluidic disposable strip 145.

In some embodiments, sensing of the microfluidic disposable strip can also be performed in a fluorescent immuno-based platform associated with a fluorescent reader unit. The fluorescent immuno-based platform and the fluorescent reader unit allow for prolonged measurement during contact lens immersion in contact lens solution in the lens container. Evolution of the colorimetric or fluorescent sensing signal relative to time is directly related to the dissolution in the contact lens solution of biomarkers from the contact lens. These biomarkers can be related to ocular surface inflammation, such as one or more of cytokines, enzymes, immunoglobulins, proteins, peptides, and lipids. The time-dependent evolution of the fluorescent sensing signal can be sent in real time to a database and prediction platform for follow up. The database and prediction platform can send back analyses and prediction results to a user interface, such as incorporated user interface on the lens container or, at the request of the user, a user interface on a smartphone, a tablet, or any other computing device. The database can also send back analyses and prediction results when the contact lens is removed from the lens container or after a selected period of time.

The solution can be analyzed by the sensor automatically, responsive to user input, or both. For example, in some embodiments, the solution can be automatically analyzed by the sensor at predetermined intervals. The predetermined time intervals can begin once a contact lens and the solution have been deposited in the cavity and/or a lid portion of the lens container is secured to the body portion. Alternatively, the predetermined intervals can begin in response to a user input on the lens container or on a device in communication with the sensor. In some embodiments, the solution can be analyzed only in response to a user input, i.e. without automatic repeated intervals.

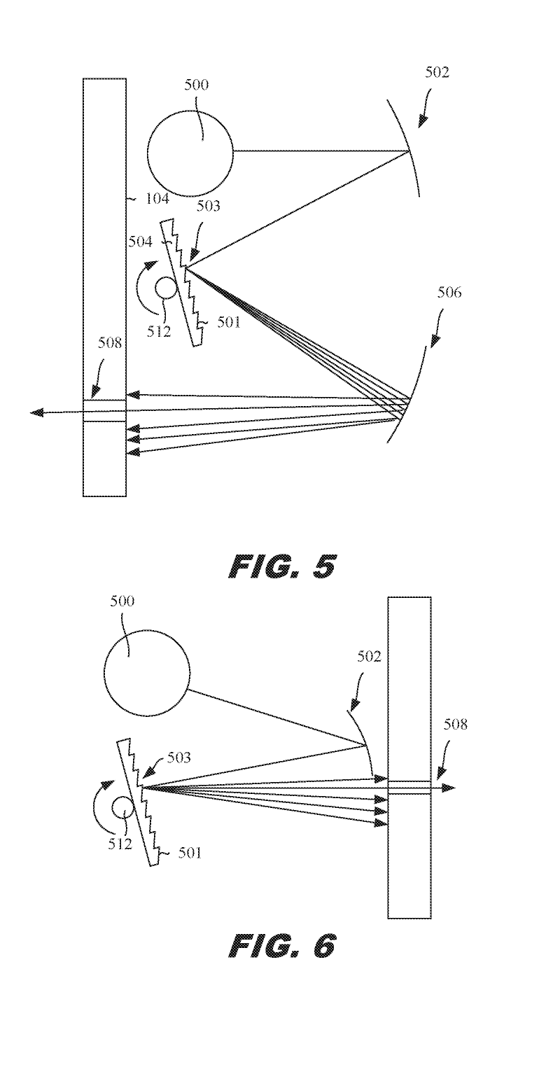

FIG. 5 depicts an example of a light transmitter 142. In this example, the light transmitter 142 includes a light source 500, a first mirror 502, a diffraction grating 504, a second mirror 506, and a slit 508 in the wall 104 of the storage container 140. In some examples, an optical window can be placed within the slit 508. In some examples, these components of the light transmitter 142 are located within the body portion of the storage container 140, the lid portion of the storage container 140, an attachment to the storage container 140, or combinations thereof.

The light source 500 can be any appropriate type of light source. In some examples, the light source is an incandescent light source, a fluorescent light source, a halogen light source, a light emitting diode light source, another type of light source, or combinations thereof. The light source can be encompassed within a blub, a diode, or another source that can be turned on and off with a switch. In some examples, the light source can emit at least two different types of wavelengths. In some situations, the light source is an infrared light source, a visible light source, an ultraviolet light source, another type of light source, or combinations thereof.

The first mirror 502 can be used to direct light from the light source 500 to the diffraction grating 504. In some examples, the first mirror 502 is curved so that different wavelengths of light come into contact with the mirror at slightly different positions and/or orientations. With the wavelengths coming off the mirror at different positions, the wavelengths also come off the first mirror at slightly different angles, which assists in causing the wavelengths to separate.

The diffraction grating 504 can be an optical component that splits light into several beams of different wavelengths in different directions. The directions of these beams depends on the spacing of the grating and the wavelength of the light. The diffraction grating 504 can be a reflective grating or a transmissive grating. In the example of FIG. 5, the diffraction grating 504 is a reflective grating that reflects the wavelengths in a way that causes the wavelengths to disperse. In this example, the diffraction grating 504 has a plurality of ridges 501 on its reflective surface 503. Each diffracted wavelength hits the reflective surface at a different location, and the angle of the diffraction grating separates the beams of different wavelengths farther apart. In other words, the diffraction grating 504 is a dispersive element that causes the wavelengths to spread out even more. In other examples with transmissive gratings, the diffraction grating 504 can be a prism that separates light into different wavelengths as the light passes through the thickness of the prism material.

In some examples, the diffraction grating 504 is connected to a tilt mechanism 512. In those examples, the tilt mechanism 512 can cause the diffraction grating 504 to move to a selectively different angle. This can cause the angle that the beams of different wavelengths come off of the diffraction grating 504 to change. In some examples, a second mirror 506 reflects the light beams off of the diffraction grating 504 towards the slit 508. In the example of FIG. 6, the diffraction grating 504 directs the light directly to the slit 508, without being directed through a second mirror.

The spacing of the light beams approaching the slit can be such that just a single beam of light passes through the optical window at a time. Thus, according to one embodiment, a single light beam is transmitted into the solution 112 at a time. To generate a light beam of a different wavelength to be transmitted through the optical window, the tilt mechanism 512 can cause the diffraction grating 504 to vary its angular position so that a different beam of a different wavelength is transmitted through the slit 508.

While these examples have depicted light transmitters with specific components in specific arrangements, the light transmitters can include more or less components than those depicted, and in different arrangements. Any appropriate type of light transmitter can be used in accordance with the principles described in the present disclosure.

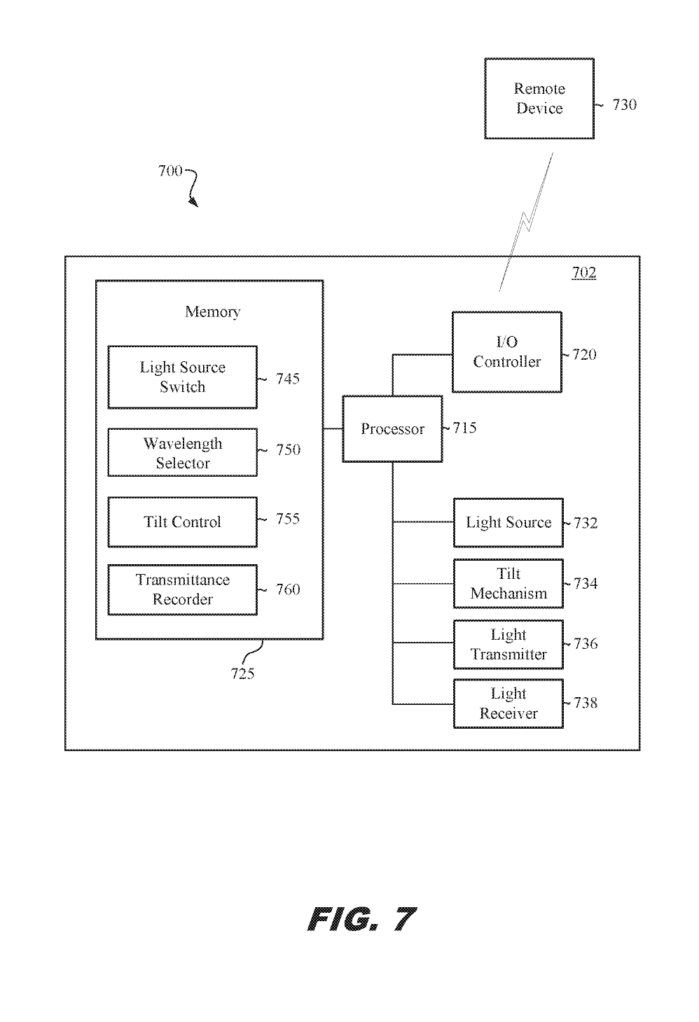

FIG. 7 depicts a diagram of an exemplary health condition system 700 incorporated into a contact lens container 702, according to one exemplary embodiment. The system 700 includes a processor 715, an I/O controller 720, and memory 725. The I/O controller 720 can be in communication with a remote device 730. The components of the system and the remote device 730 can communicate wirelessly, through hard wired connections, or combinations thereof. In some examples, the contact lens container 702 can include a transponder to communicate with the remove device 730. In some examples, the contact lens container 702 can include any number of sensors configured to detect biomarkers and/or variations to the solution in the contact lens container 702, including, but in no way limited to, sensing electrodes. In some examples, the contact lens container 702 can include a contact lens positioning system. Further, in some cases, the remove device 730 can include a base station in communication with the transponder. In some cases, the remote device 730 can be a data center. The memory 725 of the system can include a light source switch 745, wavelength selector 750, a tilt control 755, and a transmittance recorder 760. The processor 715 can also be in communication with a light source 732, a tilt mechanism 734, a light transmitter 736, and a light receiver 738. In some examples, the system can be connected to sensing electrodes.

The processor 715 can include an intelligent hardware device, (e.g., a general-purpose processor, a digital signal processor (DSP), a central processing unit (CPU), a microcontroller, an application-specific integrated circuit (ASIC), a field-programmable gate array (FPGA), a programmable logic device, a discrete gate or transistor logic component, a discrete hardware component, or any combination thereof). In some cases, the processor 715 can be configured to operate a memory array using a memory controller. In other cases, a memory controller can be integrated into the processor 715. The processor 715 can be configured to execute computer-readable instructions stored in a memory to perform various functions (e.g., functions or tasks supporting the evaluation of prescribed optical devices).

The I/O controller 720 can represent or interact with a modem, a keyboard, a mouse, a touchscreen, or a similar device. In some cases, the I/O controller 720 can be implemented as part of the processor. In some cases, a user can interact with the system via the I/O controller 720 or via hardware components controlled by the I/O controller 720. The I/O controller 720 can be in communication with any appropriate input and any appropriate output.

The memory 725 can include random access memory (RAM) and read only memory (ROM). The memory 725 can store computer-readable, computer-executable software including instructions that, when executed, cause the processor to perform various functions described herein. In some cases, the memory 725 can contain, among other things, a basic input/output system (BIOS) which can control basic hardware and/or software operation such as the interaction with peripheral components or devices.

The light source switch 745 represents programmed instructions that cause the processor 715 to switch the light source on or off. In some cases, the light source switch can block or unblock a light source that is continuously emitting light. The light source can automatically illuminate when a light portion is combined with the body portion of the contact lens container 702. In other examples, the light source switch causes the light source to illuminate when instructed to do so. The instructions to illuminate can come from the user interface, a remote device, another type of device, or combinations thereof.

The wavelength selector 750 represents programmed instructions that cause the processor 715 to select a desired wavelength of light to be transmitted through the contact lens solution. In some cases, in response to the contact lens container being instructed to analyze the contact lens solution, the wavelength selector is programmed to automatically cause the processor to start from one end of the light spectrum to the other end. In this example, the wavelength selector can sequentially test each wavelength in a consistent manner. In other examples, the wavelength selector causes only certain types of wavelengths to be tested. In situations where the contact lens solution is being tested for only a particular type of characteristic or certain types of biomarkers, at least some of the wavelengths can be omitted from the analysis. In some cases, a certain wavelength can alter or adversely affect a particular biomarker making the biomarker difficult to identify later in the analysis. In these situations, certain wavelengths can be omitted from the analysis.

The tilt control 755 represents programmed instructions that can cause the processor 715 to control the tilt mechanism that is connected to the diffraction grating. The angle at which the diffraction grating is positioned can determine which of the wavelengths is transmitted into the solution. In some examples, the wavelength selector communicates with the tilt control to cause the appropriate wavelength to be transmitted into the solution.

The transmittance recorder 760 represents programmed instructions that can cause the processor 515 to record the transmittance of the beam transmitted into the solution. In some cases, multiple beams of different transmittances are separately transmitted into the solution, and the transmittance recorder can record a transmittance for each of the wavelengths. In some cases, the transmittance recorder is in communication with the receiver that receives the light beams. In some situations, the recorder collects the transmittance strength with time stamps and the wavelengths transmitted through the solution are also timestamped. In these situations, the wavelengths transmitted can be compared to the recorded transmittance strengths based on matching times.

Correlations between certain biomarkers and their respective concentrations can go unobserved on one-on-one analysis with each of the patients. However, with such a large sample size, correlations that have been previously unobserved can be detected, for example, via data mining techniques used by the system. For example, an analysis can be run on all the biomarker characteristics of users with a specific health conditions. Such an analysis can reveal that a certain biomarkers that had not previously been linked to that health condition has a statistically significant normal concentration level, a statistically significant low concentration level, a statistically significant high concentration level, another statistically significant concentration level, a statistically insignificant type of concentration level, or combinations thereof that had not previously been observed. These correlations can help identify health conditions that can go otherwise unobserved in a patient. Even in those events where the user's health condition can be eventually diagnosed properly, comparing the obtained biomarker characteristics with the information stored in the database can result in a quicker diagnosis, or a predictive diagnosis.

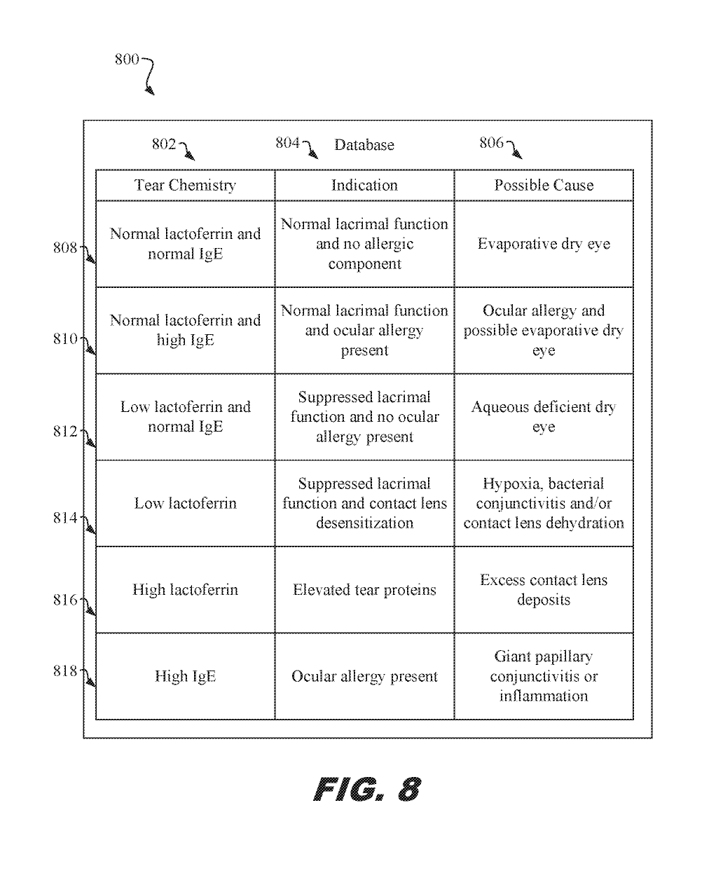

FIG. 8 depicts an example of a database 800 that associates a characteristic of the tear chemistry, potential indications, and possible causes of the tear chemistry. In this example, the database 800 includes a first column 802 that represents the tear chemistry, a second column 804 that represents the potential indications, and a third column 806 that represents the possible causes of the tear chemistry. The database 800 can include a first row 808 that includes the correlation for a tear chemistry with a normal lactoferrin level and a normal IgE level, a second row 810 that includes the correlation for a tear chemistry with a normal lactoferrin level and a high IgE level, a third row 812 that includes the correlation for a tear chemistry with a low lactoferrin level and a normal IgE level, a fourth row 814 that includes the correlation for a tear chemistry with a low lactoferrin level, a fifth row 816 that includes the correlation for a tear chemistry with a high lactoferrin level, and a sixth row 818 that includes the correlation for a tear chemistry with a high IgE level.

While the example of FIG. 8 depicts an example with the correlations of specific types of biomarkers, any appropriate type of correlation can be included in the database. In some instances, the characteristics correlated with a single biomarker can be included as depicted in rows 814, 816, 818. In other instances, the characteristics correlated with a specific set of biomarkers can be included. For example, the health conditions correlated with two or more characteristics of different types of biomarkers can be included as depicted in rows 808, 810, 812. Any appropriate number of biomarker characteristics can be included. For example, one, or alternatively, hundreds of characteristics can be collectively correlated to a specific type of health condition. Further, while the example of FIG. 8 includes specific types of biomarkers, the database can include any appropriate type of biomarker correlations.

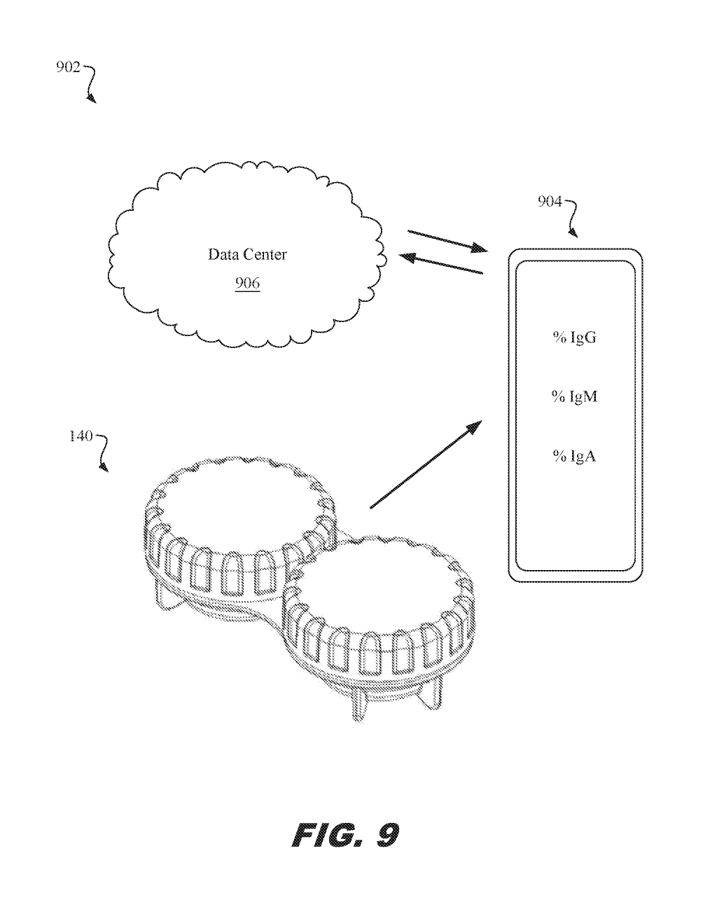

FIG. 9 depicts an example of a system 902 for determining the health condition of a user. In this example, a storage solution can be contained within a contact lens container 140. The contact lens container 140 can be in wireless communication with a mobile device 904. The mobile device 904 can relay the recorded levels to the database in the data center 906, which can send the correlations back to the mobile device 904. The mobile device 904 can present the results from the hand-held device and/or the correlations from the database in a user-interface of the mobile device. According to one exemplary embodiment, the data center 906 includes a memory component, accessible by a processor. More particularly, the data center 906 can be any physical, cloud based, or networked information and computing component, including, but in no way limited to external servers, databases, memory systems, big data aggregation, artificial intelligence, and similar database and processing systems.

At least some of the processing of the measurements obtained from the return signals from the storage solution can occur at the contact lens container 140, the mobile device 904, and/or the data center 906. In some examples, the mobile device 904 includes a program that retrieves the correlations from the database and performs additional tasks. For example, the mobile device 904 can retrieve information about the health condition from another source other than the database in response to receiving the health condition from the database. Another additional task that the mobile device 904 can perform in response to receiving the health condition is to retrieve a health professional's contact information, consult a user's calendar to set up an appointment with the health professional, schedule an appointment with the health professional, perform another task, or combinations thereof.

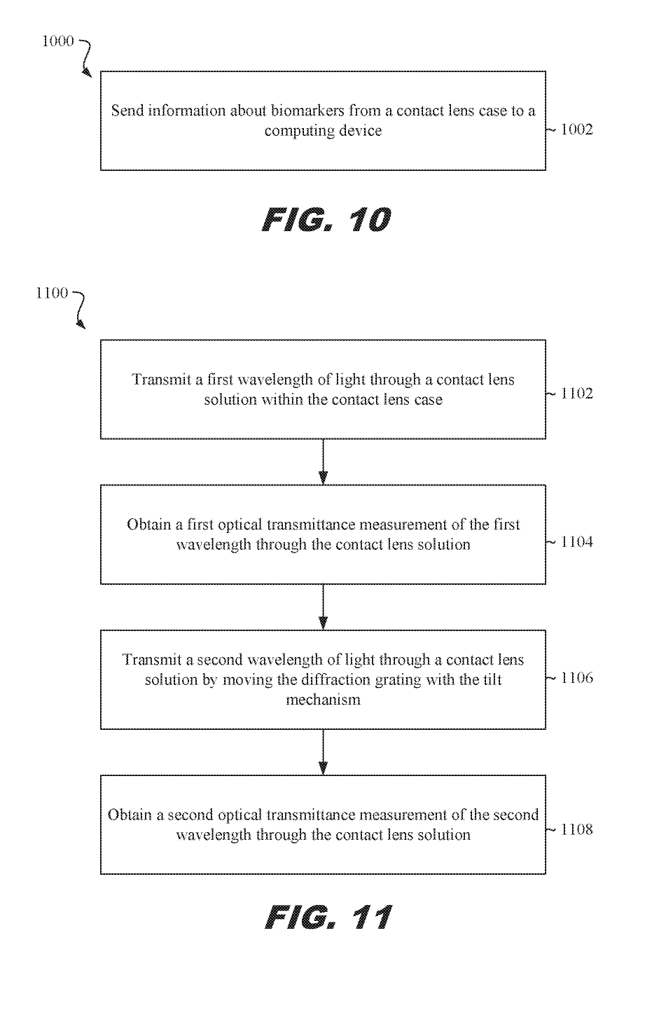

FIG. 10 illustrates an example of a method 1000 of determining a health condition. In this example, the method 1000 includes sending 1002 information about biomarkers from a contact lens container to a computing device.

At block 1002, information about the biomarkers is sent from the contact lens container to a computing device. The information can be sent to any appropriate computing device. In some examples, the computing device is a laptop, a desktop, a mobile device, a smart phone, an electronic tablet, a digital device, a remote device, a networked device, another type of device, or combinations thereof.

In some cases, the biomarkers remain on the contact lens when the biomarkers are being analyzed. In other examples, the biomarkers are removed from the contact lens before the analysis. The characteristic can include a type of biomarker, a concentration of biomarker, a location of the biomarker on the contact lens, another type of characteristic, or combinations thereof. The characteristic can involve a single biomarker. In other examples, the characteristic includes the collective condition of multiple biomarkers.

FIG. 11 illustrates an example of a method 1100 for obtaining a biomarker characteristic. In this example, the method 1100 includes transmitting 1102 a first wavelength of light through a contact lens solution within the contact lens container, obtaining 1104 a first optical transmittance measurement of the first wavelength through the contact lens solution, transmitting 1106 a second wavelength of light through a contact lens solution by moving the diffraction grating with the tilt mechanism, and obtaining 1108 a second optical transmittance measurement of the second wavelength through the contact lens solution.

In some examples, the contact lens solution includes hyaluronan, sulfobetaine, poloxamine, boric acid, sodium borate, ascorbic acid, edetate disodium, sodium chloride, hydroxyalkyl phosphate, poloxamer, sodium phosphate buffer, polyoxyethylene polyoxypropylene block copolymer with ethylene diamine, and polyaminopropyl biguanide, or combinations thereof. The contact lens can include a disinfectant, a surfactant, an anti-fungal agent, an anti-bacterial agent, another type of agent, or combinations thereof.

The removal of the biomarkers from the contact lens into the solution can occur over any appropriate time period. In some examples, the biomarkers are in the solution for at least one minute, at least five minutes, at least 20 minutes, at least 45 minutes, at least an hour, at least two hours, at least 5 hours, at least 7 hours, at least one day, at least two days, another appropriate time period, or combinations thereof.

In some examples, the contact lens is free of surface cavities that are constructed to be binding sites for biomarkers or to draw in tear fluid into the contact lens. In some examples, the contact lens is free of surface treatments that target the binding of specific biomarkers to the contact lens.

In some examples, the storage solution includes binding agents that are configured to facilitate the bonding between a surface of the contact lens and a biomarker from the tear fluid. In other examples, no binding agents are introduced to the contact lens solution. The contact lens can include a surface where the biomarkers are as likely to bind to any surface of the contact lens as any other surface of the contact lens. In some examples, the biomarkers can attach to the optical zone of the contact lens, a peripheral zone of the contact lens, an edge of the contact lens, a posterior side of the contact lens, an anterior side of the contact lens, another area of the contact lens, or combinations thereof.

The contact lens can be made through any appropriate manufacturing method. In some examples, the contact lenses are molded into their shape. In other examples, the contact lenses are machined to their precise shape. In yet other examples, the contact lens are cast molded or spin cast. Spin cast contact lenses can make a continuous surface on the posterior side of the contact lens that matches a profile constructed to assist the user with his or her vision. The front side of the contact lens during a spin casting procedure can include a profile that matches a contact lens mold. The contact lens mold can include a continuous, curved surface without interruptions. In some examples, the spin cast contacts lens provide for a continuous surface that is substantially free of interruptions, such as micro-cavities. In some examples, having a continuous, interruption free surface on both the anterior side and the posterior side can prevent the collection of tear fluid in the contact lens. Avoiding the collection of tear fluid can prevent the contact lens from having an additional amount of weight. Further, when the contact lens is introduced into the solution, a substantial amount of tear fluid may not mix with the contact lens solution, which can skew the volume of fluid in being analyzed and affect the concentration analyses. In some examples where tear fluid is not collected, just the biomarkers can be carried with the contact lens into the solution. Thus, the analysis does not have to be adjusted to accommodate an increase in fluid. However, in some examples, the amount of fluid being analyzed may not require a precise amount of fluid. In one example, the contact lens container can include a fill line and the measurements performed by the sensor can be adequate enough if the solution is close to being at the fill line, but not required to be precisely at the fill line. Further, by not modifying the contact lens to have an enhanced ability to collect specific biomarkers, the concentrations of the biomarkers that bind to the contact lens can be more reflective of the actual concentration of that biomarker in the tear fluid. An enhanced ability to collect a particular biomarker or a wide variety of biomarkers can cause a disproportionate amount of that biomarker to bind to the contact lens, which can skew the measurement levels made when analyzing the solution and potentially lead to an inaccurate characterization of the actual concentration of the biomarker.

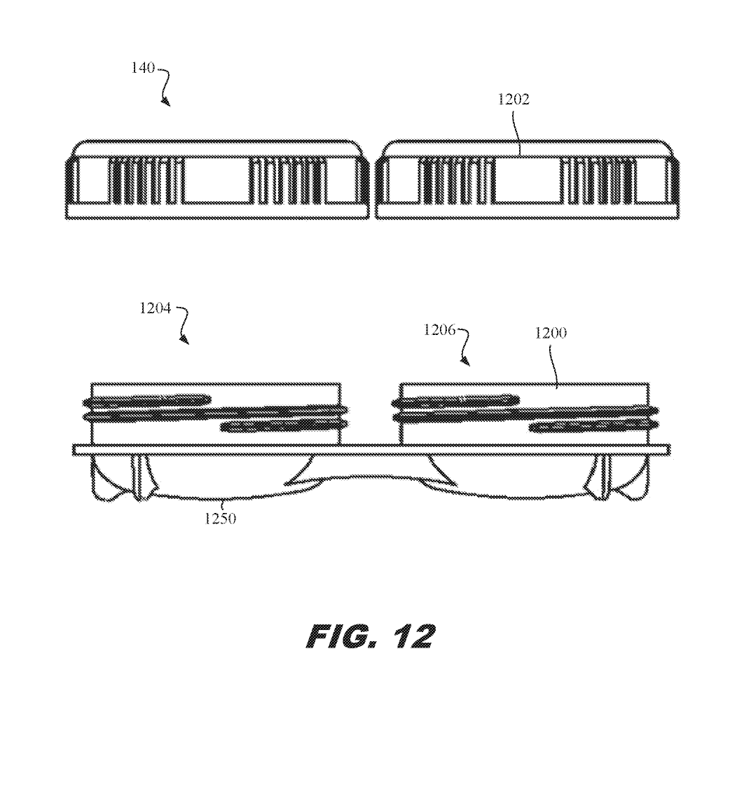

FIGS. 12-17 illustrate various contact lens containers, according to the present exemplary teachings. Each of the contact lens containers can include the elements disclosed in connection with FIG. 7, including a processor, memory, and I/O controller, and the like.

FIG. 12 depicts an example of a contact lens container 140. In this example, the container 140 includes a body portion 1200 and a plurality of lid portions 1202. Each of the lid portions 1202 can interlock with the body portion 1200. In the illustrated example, the lid portion 1202 can threadedly interlock with the body portion 1200.

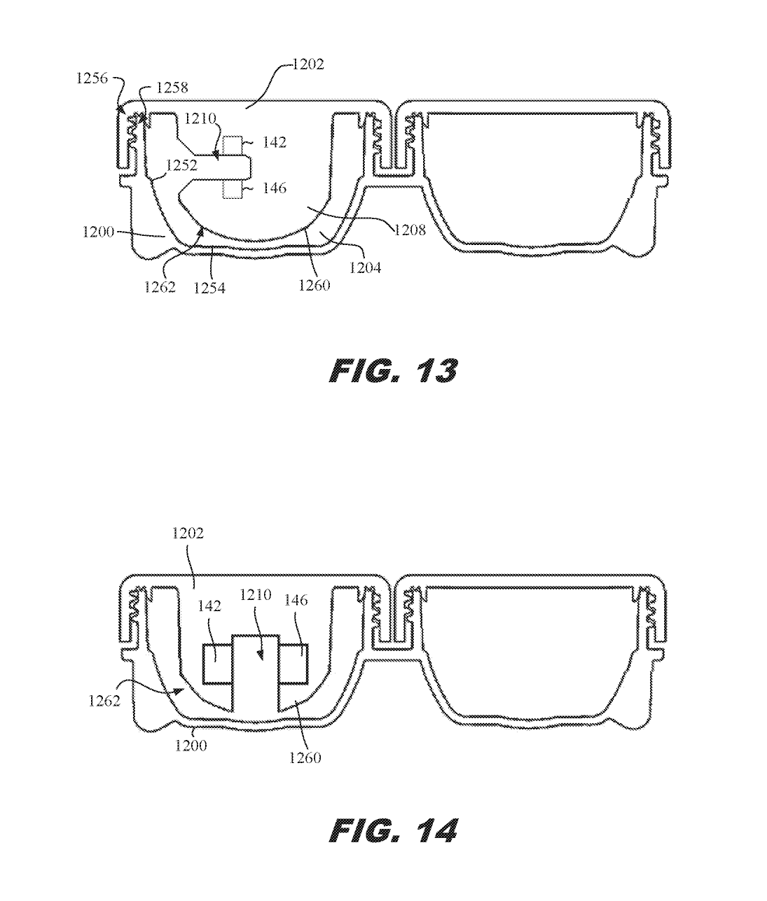

The body portion 1200 can have a substantially flat undersurface 1250 that provides stability to the container 140 when resting on a support surface, such as a counter top or sink surface. In other examples, the body portion 1200 includes a plurality of legs that stabilize the body portion 1200 in an upright orientation. In the upright position, the contact lens container 140 is oriented so that the storage solution pools in the bottom of the cavity away from the threaded portions or other connection mechanisms that secure the lid portion 1202 to the body portion 1200. The body portion 1200 can also include an inner wall (FIG. 13, 1252) that is connected to a floor (FIG. 13, 1254). The inner wall 1252 and the floor 1254 collectively define the cavity. The cavity can be configured to receive a volume of contact lens storage solution. A contact lens can be inserted into the cavity into the storage solution for a desired period of time, such as overnight, until the user decides to reinsert the contact lens back into the user's eye.

The body portion 1200 can include a first cavity 1204 and a second cavity 1206. Since a user generally wears a separate contact lens in each eye, the contact lens container 140 can include the first cavity 1204 for the first contact lens and the second cavity 1206 for the second contact lens. A sensor can be incorporated into each of the cavities, or just one of the cavities. In some examples, the biomarker profile of one eye can be similar or the same to the biomarker profile of the other eye. In these examples, testing the biomarkers of one eye can be sufficient to understand the user's tear's chemistry. However, in other examples, testing each of the eye's tear fluid can help identify profiles that may not be realized when testing just a single eye.

Any appropriate storage solution can be used in connection with the principles disclosed herein. In some examples, the storage solution includes a disinfectant that kills bacteria, viruses, fungus, germs, enzymes, undesirable organisms, or combinations thereof that are on the contact lens. In some examples, the storage solution also prevents a protein build-up, a lipid build-up, a debris build-up, or other type of build-up on the contact lens. Further, the storage solution can include ingredients that improve wettability and comfort of silicon hydrogel contact lenses or other types of contact lens. In some cases, the storage solution includes a saline solution, a hydrogen peroxide solution, another type of solution, or combinations thereof.

The contact lens container 140 can be formed through any appropriate mechanism. In some cases, the contact lens container 140 is injection molded using synthetic resins, such as polypropylene (PP), polyethylene (PE), polystyrene (PS), polycarbonate (PC), polyethylene terephthalate (PET), acrylonitrile butadiene styrene copolymer (ABS), propylene ethylenic copolymer, or combinations thereof. In other examples, the contact lens container 140 can be casted, machined, or otherwise formed. In some cases, the lid portion 1202 is made of the same materials as the body portion 1200.

In some cases, the body portion 1200 includes a first thread portion (FIG. 13, 1256), and the lid portion 1202 includes a second thread portion (FIG. 13, 1258). In some cases, the first thread portion 1256 is an outer thread portion, and the second thread portion 1258 is an inner thread portion. However, in other examples, the first thread portion 1256 is an inner thread portion, and the second thread portion 1258 is an outer thread portion. The first thread portion 1256 and the second thread portion 1258 can be threadedly connected to one another. With the lid portion 1202 secured to the body portion 1200 through the threaded portions, the lid portion 1202 closes off the cavity.

While these examples have been described with reference to the contact lens container 140 having the lid portion 1202 and the body portion 1200 connected through complementary threaded portions, the lid portion 1202 and the body portion 1200 can be connected through any appropriate mechanism. For example, the lid portion 1202 and the body portion 1200 can be secured together through a snap connection, a compression fit connection, a hinged connection, another type of connection, or combinations thereof. In some examples, the connection is water tight to prevent the storage solution from leaking out of the cavity when the contact lens container 140 is oriented on its side or is oriented upside-down.

FIGS. 13 and 14 depict example layout diagrams of a sensor incorporated into a lid portion 1202. In these examples, the lid portion 1202 includes a protrusion 1208 that protrudes into a volume of the first cavity 1204. The protrusion 1208 can have a cross-sectional thickness that is less than the cross-sectional thickness of the cavity, thereby allowing fluid to move within the space between the surface of the cavity's walls and the surface of the protrusion 1208. The protrusion can also be sized to pin a contact lens to the bottom of the cavity, or at least cause the contact lens to be located between the bottom of the cavity and a distal end of the protrusion 1208.

A protrusion 1208 can be connected to the lid portion 1202. The protrusion 1208 can extend farther away from the lid portion 1202 than the second threaded portion. The protrusion 1208 can include a distal end 1260, and the distal end 1260 can include a curved surface 1262.

In some cases, a center portion of the contact lens comes into contact with a central portion of the floor 1254 of the cavity. In those examples where a gap between the floor 1254 and the distal end 1260 of the protrusion 1208 are smaller than the sagittal depth of the contact lens, the protrusion 1208 and the floor 1254 can collective impose a compressive load on the contact lens that assists in keeping the contact lens against the curved surface 1262. However, due to the curvature of the distal end 1260, the gap can progressively increase from the central portion of the floor 1254 towards the edge of the curved surface 1262. In such circumstances, the contact lens can otherwise be prone to dislodging from the curved surface 1262 if the contact lens is positioned off center on the curved surface 1262.

The protrusion 1208 can cause the contact lens to be located in a space within the cavity's volume that is away from a light beam that can be transmitted by the sensor. In other words, the protrusion 1208 can assist in locating the contact lens in a region of the cavity, so that the contact lens is less likely to interfere with the measurements taken in the contact lens solution.

A channel 1210 can be defined in the protrusion 1208 that is sized to allow a portion of the contact lens solution to enter within a volume defined by the channel 1210. The light transmitter 142 and the light receiver 146 can be located proximate to the channel 1210 so that they can test the contact lens solution that is located in the channel 1210. In the example of FIG. 13, the channel 1210 is defined in the side wall of the protrusion 1208, and in FIG. 14, the channel 1210 is formed in the distal end 1260 of the protrusion 1208. In the example of FIG. 14, to avoid trapping air within the channel 1210 as the protrusion 1208 comes into the contact lens solution, a vent hole (not shown) can connect the channel to the surface of the protrusion's wall.

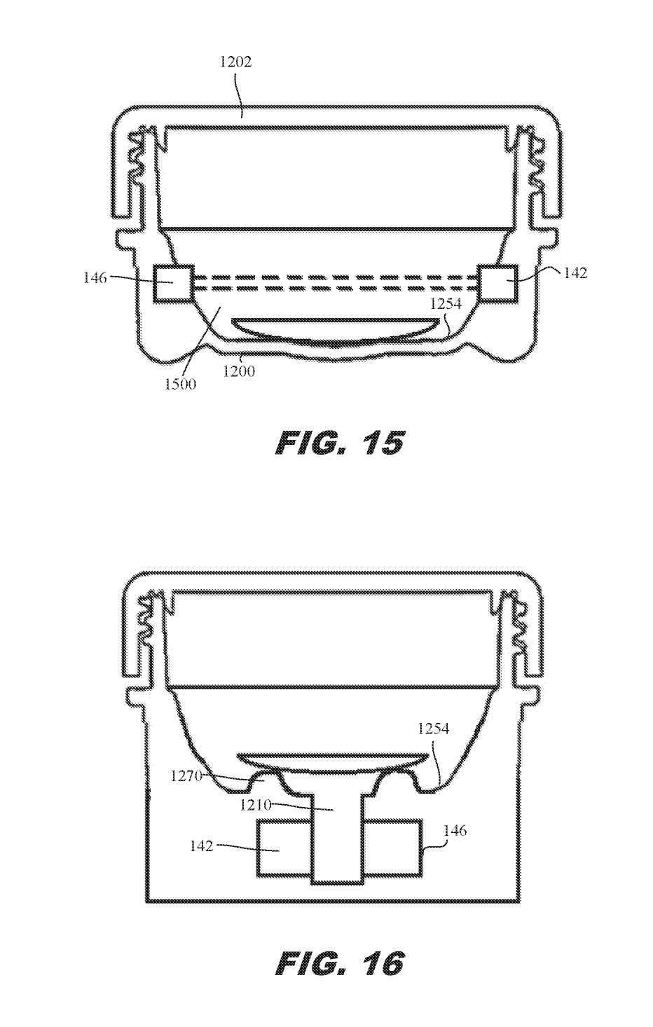

FIG. 15 depicts an example of a cavity 1500 within the body portion 1200 that has a light receiver and a light transmitter 142 located at a distance from the floor 1254 of the cavity to be above a contact lens that settles to the bottom of the cavity.

FIG. 16 depicts an example of the channel 1210 defined in the floor 1254 and the associated light transmitter 142 and receiver 146 are adjacent to the channel 1210 where the solution can be analyzed. In the illustrated example, risers 1270 protrude from off of the floor 1254 to space the contact lens off of the floor 1254. The risers 1270 are spaced so that the storage solution can pass around the risers 1270. Thus, the risers 1270 prevent the contact lens from blocking the solution from entering into the channel 1210.

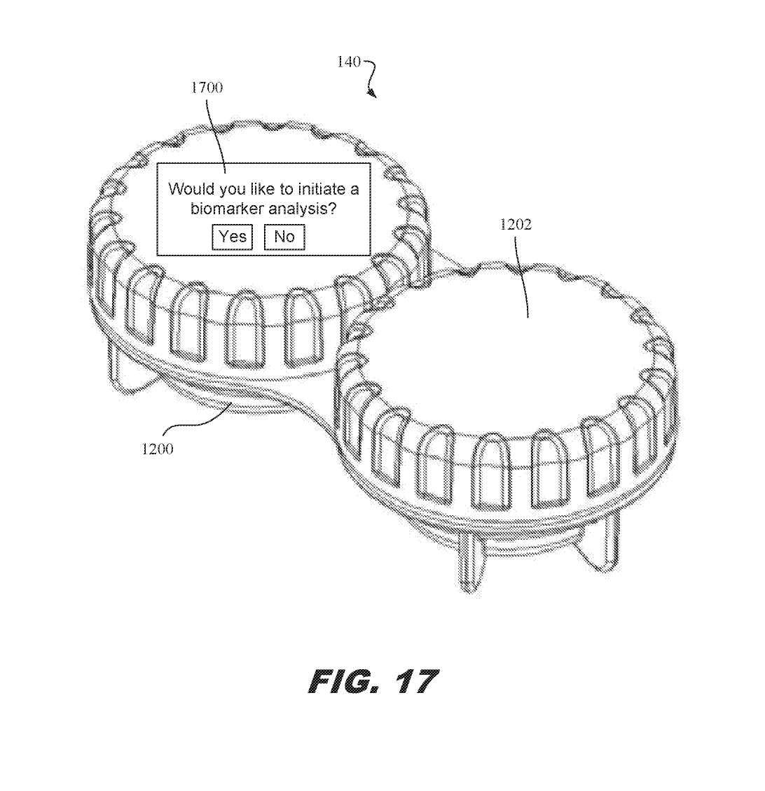

FIG. 17 depicts an example of a contact lens container 140 with a user interface 1700 disposed thereon. The user interface 1700 can be used to present messages to the user or present options to the user. The user can also use the user interface 1700 to give instructions to the contact lens container 140. In other examples, the user interface is incorporated into a device that is in communication with the contact lens container 140. For example, the contact lens container 140 can be in wireless communication with a mobile device, and the user interface of the mobile device can operate as an interface between the container and the user. In other examples, the user interface can be hardwired to the container 140. A non-exhaustive list of devices that can be in communication with the container and provide a user interface include, but are not limited to, a mobile device, a smart phone, an electronic tablet, a laptop, a desktop, a computing device, a networked device, another type of device, or combinations thereof.

While the example of FIG. 17 depicts the user interface 1700 incorporated into the lid portion 1202 of the container 140, the user interface 1700 can be incorporated into any appropriate portion of the container 140. For example, the user interface 1700 can be incorporated into the body portion 1200, the side of the container, the undersurface of the container, another portion of the container, or combinations thereof.

Examples of messages that can be presented to the user include the results of the analysis, an option to initiate the analysis, a request to change batteries, a request to replace the storage solution, a schedule of when the analysis is to be performed, an option to test for specific health conditions, a request to tighten the lid portion, a request to replace a light source or another component of the container, a request to insert the contact lens, a request for permission to send the results of a test to remote device, another type of message, or combinations thereof.

Examples of instructions that the user can communicate to the container through the user interface includes initiating a test, restricting the testing to specific types of conditions, limiting the range of wavelengths, setting a time to cause a test to be run, sending the test results to a remote device, to discontinue a test, to not perform a test, another type of instructions, or combinations thereof. A number of test examples are detailed below.

### EXAMPLES AND METHODOLOGIES

### Example 1

In one example, one or more protein biomarkers previously deposited on a contact lens were detected using near-infrared spectroscopy. The sensor included a near-infrared spectrometer having a light transmitter and a light receiver incorporated into a body of a lens container. The light transmitter (or radiation source) was positioned in the body of the lens container to direct light substantially perpendicular to a contact lens when the contact lens was stored in the standard position in the lens container during overnight removal. The contact lens container lid portion was closed when the light transmitter was transmitting the light, thus containing the light within the lens container.

The light transmitter provided light having a wavelength in the region of 900 nm to 2500 nm. For example, the light transmitter was configured to provide light having a wavelength in a narrower selected region of 1300 nm to 1600 nm, including a first overtone of water. The light transmitter could repeatedly emit a spectra in response to a user request. Additionally or alternatively, the light transmitter could be programmed to automatically repeat emission of a spectra of light from the light transmitter. For example, spectral analysis could be performed every hour after the contact lens was inserted into the lens container and the container lid portion was closed. Spectral analysis can be performed 1 hour after the contact lens was inserted into the lens container, 2 hours after the contact lens was inserted into the lens container, and/or after overnight incubation in the contact lens solution in the lens container. Spectral analysis could be initiated using a button or actuator on the lid portion. Spectral analysis could also be initiated wirelessly via a user interface on a smartphone or a tablet. Results of the spectral analysis were temporarily stored in the memory unit and analyzed in the processing units incorporated into the body portion.

Once processed, the spectral results were sent to a database. Algorithms developed for ocular health condition prediction analyze data of the spectral results and compare the spectral results with the health database of the patient. A database and computing platform send back an analysis and prediction results to a user interface such as the user interface on a smartphone or tablet.

### Example 2

In another example, one or more biomarkers previously deposited on a contact lens are detected using a microfluidic disposable strip. The microfluidic disposable strip is incorporated into the body portion of the lens container. When the cavity is at least partially filled with a contact solution and the contact lens is in the standard position in the lens container, the microfluidic strip sensor continuously collects contact lens solution and solutes dissolved from worn contact lens.

Sensing of the microfluidic disposable strip is performed in an immuno-based platform with colorimetric reading. An ultraviolet spectrometer incorporated into the body of the lens container is used to quantitatively analyze a color change of a substrate on the microfluidic disposable strip to target specific antibodies on the microfluidic disposable strip.

Sensing of the microfluidic disposable strip also is performed in a fluorescent immuno-based platform associated with a fluorescent reader unit. The fluorescent immuno-based platform and fluorescent reader unit allow for prolonged measurement during contact lens immersion in contact lens solution in the lens container. Colorimetric evolution of the fluorescent sensing signal with time is directly related to the dissolution in the contact lens solution of biomarkers from the contact lens. These biomarkers can be related to ocular surface inflammation, such as one or more of cytokines, enzymes, immunoglobulins, proteins, peptides, and lipids. The time-dependent evolution of the fluorescent sensing signal is sent in real time to a database and prediction platform for follow-up.

The database and prediction platform send back analyses and prediction results to a user interface, such as incorporated user interface on the lens container or, at the request of the user, a user interface on a smartphone or tablet. The database also sends back analyses and prediction results when the contact lens is removed from the lens container or after a selected period of time.

### Example 3

In another example, various dilutions of glucose water were analyzed to demonstrate detection of exemplary particles in a solution. Sample solutions were prepared having the following concentrations: pure water, 1 gram glucose per liter of water, 5 grams glucose per liter of water, 10 grams glucose per liter of water, 50 grams glucose per liter of water, and 100 grams glucose per liter of water.

For each measurement, several drops of the sample solution were placed on a diamond crystal sample plate. Infrared absorbance at 400-4000 cm-1 of each of the samples was measured with a Thermo Scientific Nicolet 8700 FT-IR spectrometer, based on the following measurement conditions: attenuated total reflectance (ATR) reflection measurement method; diamond crystal sample plate; 256 seconds measurement time; approximately 1.9 cm-1 measurement interval; approximately 4 cm-1 resolution; and atmospherically and background corrected by ATR.

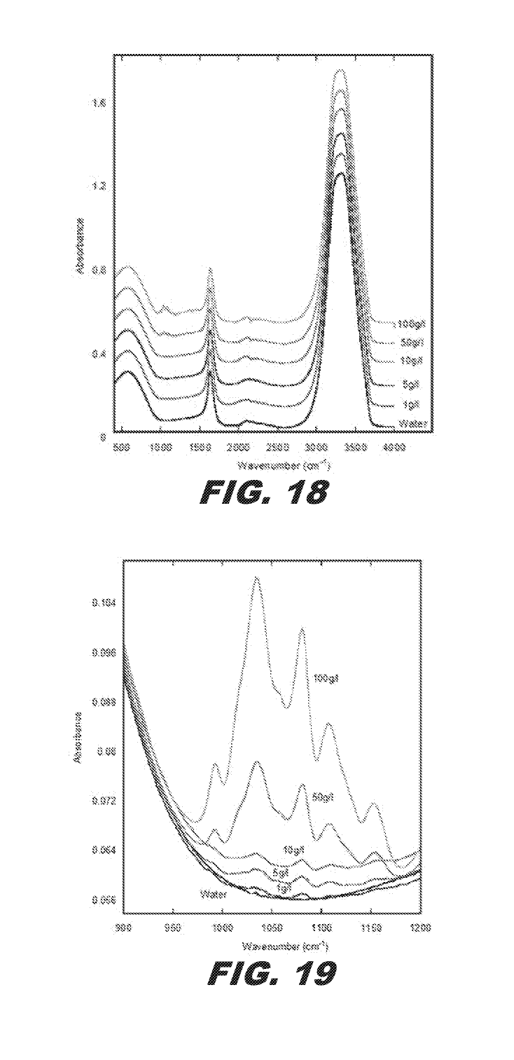

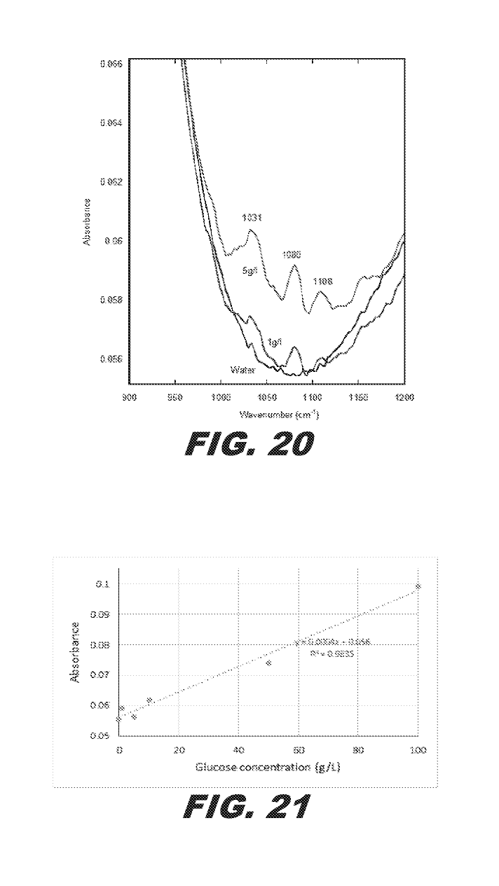

FIG. 18 is a graph showing infrared absorbance from 400 to 4000 cm-1 of the six samples. In the graph of FIG. 18, a first region from approximately 900 to 1200 cm-1 and a second region from approximately 2000 to 2300 cm-1 demonstrate concentration-dependent regions among the six sample. The first region and the second region, then, are likely regions specific to the glucose molecule. FIG. 19 is a graph showing infrared absorbance in the first region referenced above, from 900 to 1200 cm-1, of the six samples. FIG. 19 demonstrates that the spectra change depends on the concentration of glucose. FIG. 20 is a more detailed graph showing infrared absorbance in the first region referenced above, from 900-1200 cm-1, of the lower concentration samples of pure water, 1 gram glucose per liter of water, and 5 grams glucose per liter of water. Peaks are shown in FIG. 20 at wavelengths of 1031 cm⁻¹, 1080 cm⁻¹, and 1108 cm⁻¹. A calibration curve, then, can be set at 1080 cm-1 for estimation of glucose concentration in a sample. FIG. 21 shows a calibration curving using an absorbance of 1080 cm-1, which can be used to provide the concentration of a new sample when absorbance of the new sample is provided.

In one embodiment, disclosed herein, an ophthalmic lens container, includes a body, the body including a floor, at least one wall connected to the floor, a cavity at least partially defined by the floor and the at least one wall, and a sensor oriented to take a measurement of a lens solution within the cavity when the cavity is at least partially filled with the lens solution.

The ophthalmic lens container can include the sensor incorporated into the body. In one embodiment, at least a portion of the sensor is exposed within the cavity. In one embodiment, the sensor includes a sensing electrode or an optical spectrometer. In one embodiment, the sensor includes an energy source. The energy source can be a light transmitter, an acoustic energy source, or a radiation source. The light transmitter can be a mirror, an optical window, a diffraction grating, or a light source.

In one embodiment, the energy source is exposed within the cavity. According to one embodiment, the sensor further includes a receiver oriented to detect a characteristic of energy from the at least one energy source from within the lens solution. The receiver can be on a side in the cavity opposite the energy source or located on a same side in the cavity as the energy source. The ophthalmic lens container can include a lid shaped to interlock with the body. In one embodiment, the sensor is incorporated into the lid. Additionally, according to one embodiment, the lens container includes a user interface in communication with the sensor. In one embodiment, the user interface is incorporated into the body.

In one embodiment, the ophthalmic lens container includes a user interface that is in wireless communication with the sensor. In one embodiment, the user interface is configured to present an option to initiate an analysis of the lens solution. In another embodiment, the user interface is configured to present an option to schedule an analysis of the lens solution. In one embodiment, the user interface is configured to present an option to send the measurement to a remote device. The ophthalmic lens container of claim can include a user interface that is configured to present a result of the measurement.

In on embodiment, the ophthalmic lens container includes a sensor that is configured to take separate measurements at different energy intensities. In one embodiment, taking separate measurements at different energy intensities includes taking measurements at different optical wavelengths. In one embodiment the sensor includes a near infrared spectrometer configured to provide light in a wavelength region of approximately 900 nm to approximately 2500 nm in the cavity. In one embodiment, the near infrared spectrometer includes a light source directed substantially perpendicular to a lens when a lens is deposited into the cavity. The near infrared spectrometer is configured, in one embodiment, to provide the light in a wavelength region of approximately 1300 nm to approximately 1600 nm.

In one embodiment, the sensor is configured to emit and absorb the light in response to a user request. The sensor can be in wireless communication with an electronic device, and the user request is received from the electronic device. The sensor can be configured to repeatedly emit and absorb the light automatically in predetermined intervals. The predetermined intervals can be every hour after a lens is deposited into the cavity until the lens is removed from the cavity.

In one embodiment, the sensor includes a microfluidic disposable strip secured to the body, the microfluidic disposable strip configured to collect a portion of the lens solution and one or more solutes dissolved in the lens solution from a lens placed in the cavity with the lens solution. In one embodiment, the microfluidic disposable strip includes a substrate having an immuno-based platform configured to change colors responsive to one or more biomarkers collecting on the microfluidic disposable strip, and the sensor further includes an ultraviolet spectrometer configured to quantitatively analyze a color change of the substrate on the microfluidic disposable strip. Alternatively, the microfluidic disposable strip includes a substrate having a fluorescent immuno-based platform configured to change colors responsive to one or more biomarkers collecting on the microfluidic disposable strip, and the sensor further comprises a fluorescent reader unit configured to quantitatively analyze a color change of the substrate on the microfluidic disposable strip.

In one embodiment, the one or more biomarkers are associated with ocular surface inflammation and include at least one of a cytokine, an enzyme, an immunoglobulin, a protein, a peptide, or a lipid.

The lens container can include a contact lens case. Alternatively, the lens container is configured to receive an inner ocular lens (IOL).

According to one exemplary embodiment, a contact lens container includes a body, the body including a floor, at least one wall connected to the floor, a cavity defined, at least in part, by the floor and the at least one wall, and a light transmitter incorporated into the contact lens container and oriented to transmit a light into the cavity.

In one example, the contact lens container includes a lid shaped to interlock with the body. In one example, the light transmitter is incorporated into the lid. Alternatively, the light transmitter is incorporated into the body.

In one embodiment, the light transmitter operates within a range that includes a least a portion of a visible light range. In another embodiment, the light transmitter operates within a range that includes a least a portion of an infrared light range. In another embodiment, the light transmitter operates within a range that includes a wavelength region of approximately 1300 nm to approximately 1600 nm. In one embodiment, the light transmitter operates within a range that comprises a least a portion of an ultraviolet light range.

The exemplary contact lens container also includes, according to one embodiment, a light receiver positioned proximate to the cavity to receive at least a portion of light from the light transmitter.

In one embodiment, the contact lens container includes a light receiver positioned within the cavity to receive at least a portion of light from the light transmitter.

In one embodiment, the contact lens container further includes a microfluidic disposable strip secured to the body, the microfluidic disposable strip configured to collect a portion of the contact lens solution and one or more solutes dissolved in the contact solution from a contact lens placed in the cavity with the contact lens solution. In one embodiment, the microfluidic disposable strip includes a substrate having an immuno-based platform configured to change colors responsive to one or more biomarkers collecting on the microfluidic disposable strip, and the light transmitter includes an ultraviolet spectrometer configured to quantitatively analyze a color change of the substrate on the microfluidic disposable strip. In one embodiment, the microfluidic disposable strip includes a substrate having a fluorescent immuno-based platform configured to change colors responsive to one or more biomarkers collecting on the microfluidic disposable strip, and the light transmitter further includes a fluorescent reader unit configured to quantitatively analyze a color change of the substrate on the microfluidic disposable strip.

The contact lens container, in one embodiment, the disposable strip detects one or more biomarkers that are associated with ocular surface inflammation and include at least one of a cytokine, an enzyme, an immunoglobulin, a protein, a peptide, or a lipid.

In one embodiment, the contact lens container further includes a sensing electrode disposed within the contact lens container.

In one embodiment, a contact lens container includes a body, the body including a floor, at least one wall connected to the floor, a cavity defined, at least in part, by the floor and the at least one wall, and a light transmitter incorporated into the contact lens container and oriented to transmit a light within the cavity.

In one embodiment, a method of ascertaining a health condition of a user, includes sending information about biomarkers from a contact lens to a computing device. In one embodiment, the information is obtained at least by measuring an optical transmittance through a contact lens solution. In one embodiment, the information is obtained at least by measuring a first optical transmittance at a first wavelength through a contact lens solution and a second optical transmittance at a second wavelength through the contact lens solution. In one embodiment, the first wavelength and the second wavelength are within an infrared range of wavelengths.

In one embodiment, the information is sent from a contact lens container, where the contact lens container includes a light source, and a diffraction grating positioned to diffract light from the light source. According to one embodiment, the contact lens container further includes a tilt mechanism attached to the diffraction grating. Additionally, the contact lens container can further include a slit positioned to receive at least some of the light from the light source after being diffracted with the diffraction grating, wherein the tilt mechanism is configured to different a range of wavelengths through the slit by positioning the diffraction grating.

The method can further include transmitting a first wavelength of light through a contact lens solution within the contact lens container, obtaining a first optical transmittance measurement of the first wavelength through the contact lens solution, transmitting a second wavelength of light through the contact lens solution by moving the diffraction grating with the tilt mechanism, and obtaining a second optical transmittance measurement of the second wavelength through the contact lens solution.

In one embodiment, the method can include analyzing a microfluidic disposable strip in the contact solution to obtain the information. The microfluidic disposable strip can be analyzed with a ultraviolet spectrometer. In one embodiment, the microfluidic disposable strip is analyzed with a fluorescent reader unit.

Unless otherwise indicated, all numbers or expressions, such as those expressing dimensions, physical characteristics, etc., used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

In addition, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

We claim:

1. An ophthalmic lens container, comprising:
a body, the body including:
a floor;
at least one wall connected to the floor;
a cavity at least partially defined by the floor and the at least one wall; and
a sensor oriented to take a measurement of a lens solution within the cavity when the cavity is at least partially filled with the lens solution.

2. The ophthalmic lens container of claim 1, wherein the sensor is incorporated into the body.

3. The ophthalmic lens container of claim 1, wherein at least a portion of the sensor is exposed within the cavity.

4. The ophthalmic lens container of claim 3, wherein the sensor comprises one of a sensing electrode, an energy source, or an optical spectrometer.

5. The ophthalmic lens container of claim 4, wherein the energy source comprises one of a light transmitter, an acoustic source, and a radiation source.

6. The ophthalmic lens container of claim 5, wherein the light transmitter comprises one of a mirror, an optical mirror, a diffraction grating, and a light source.

7. The ophthalmic lens container of claim 4, wherein the sensor further comprises a receiver oriented to detect a characteristic of energy from the at least one energy source from within the lens solution.

8. The ophthalmic lens container of claim 7, wherein the receiver is on a side in the cavity opposite the energy source or on a same side in the cavity as the energy source.

9. The ophthalmic lens container of claim 1, further comprising a lid shaped to interlock with the body;
wherein the sensor is incorporated into the lid.

10. The ophthalmic lens container of claim 1, further comprising a user interface in communication with the sensor, wherein the user interface is incorporated into one of the body or a lid shaped to interlock with the body.

11. The ophthalmic lens container of claim 10, wherein the user interface is configured to present at least one of an option to initiate an analysis of the lens solution, an option to schedule an analysis of the lens solution, an option to send the measurement to a remote device, or to present a result of the measurement.

12. The ophthalmic lens container of claim 1, wherein the sensor is configured to take separate measurements at different energy intensities.

13. The ophthalmic lens container of claim 12, wherein taking separate measurements at different energy intensities includes taking measurements at different optical wavelengths.

14. The ophthalmic lens container of claim 1, wherein the sensor comprises a near infrared spectrometer configured to provide light in a wavelength region of approximately 900 nm to approximately 2500 nm in the cavity.

15. The ophthalmic lens container of claim 14, wherein:
the near infrared spectrometer comprises a light source directed substantially perpendicular to a lens when a lens is deposited into the cavity;
the near infrared spectrometer is configured to provide the light in a wavelength region of approximately 1300 nm to approximately 1600 nm;
the sensor is configured to emit and absorb the light in response to a user request; and
the sensor is in wireless communication with an electronic device, and the user request is received from the electronic device.

16. The ophthalmic lens container of claim 15, wherein the sensor is configured to repeatedly emit and absorb the light automatically in predetermined intervals.

17. The ophthalmic lens container of claim 1, wherein the sensor comprises a microfluidic disposable strip secured to the body, the microfluidic disposable strip configured to collect a portion of the lens solution and one or more solutes dissolved in the lens solution from a lens placed in the cavity with the lens solution;
wherein:
the microfluidic disposable strip comprises a substrate having an immuno-based platform configured to change colors responsive to one or more biomarkers collecting on the microfluidic disposable strip; and
the sensor further comprises an ultraviolet spectrometer configured to quantitatively analyze a color change of the substrate on the microfluidic disposable strip.

18. The ophthalmic lens container of claim 1, wherein the sensor comprises a microfluidic disposable strip secured to the body, the microfluidic disposable strip configured to collect a portion of the lens solution and one or more solutes dissolved in the lens solution from a lens placed in the cavity with the lens solution, wherein:
the microfluidic disposable strip comprises a substrate having a fluorescent immuno-based platform configured to change colors responsive to one or more biomarkers collecting on the microfluidic disposable strip; and
the sensor further comprises a fluorescent reader unit configured to quantitatively analyze a color change of the substrate on the microfluidic disposable strip.

19. The ophthalmic lens container of claim 18, wherein the one or more biomarkers are associated with ocular surface inflammation and include at least one of a cytokine, an enzyme, an immunoglobulin, a protein, a peptide, or a lipid.

20. A contact lens container, comprising:
a body, the body including:
a floor;
at least one wall connected to the floor;
a cavity defined, at least in part, by the floor and the at least one wall;
a lid shaped to interlock with the body; and
a light transmitter incorporated into the contact lens container and oriented to transmit a light into the cavity.

21. The contact lens container of claim 20, wherein the light transmitter is incorporated into one of the lid and the body; and
wherein the light transmitter operates within a range that includes a least a portion of a visible light range.

22. The contact lens container of claim 21, wherein the light transmitter operates within a range that comprises a wavelength region of approximately 1300 nm to approximately 1600 nm.

23. The contact lens container of claim 20, further comprising a microfluidic disposable strip secured to the body, the microfluidic disposable strip configured to collect a portion of the contact lens solution and one or more solutes dissolved in the contact solution from a contact lens placed in the cavity with the contact lens solution.

24. The contact lens container of claim 23, wherein:
the microfluidic disposable strip comprises a substrate having an immuno-based platform configured to change colors responsive to one or more biomarkers collecting on the microfluidic disposable strip; and
the light transmitter comprises an ultraviolet spectrometer configured to quantitatively analyze a color change of the substrate on the microfluidic disposable strip.

25. The contact lens container of claim 23, wherein:
the microfluidic disposable strip comprises a substrate having a fluorescent immuno-based platform configured to change colors responsive to one or more biomarkers collecting on the microfluidic disposable strip; and
the light transmitter further comprises a fluorescent reader unit configured to quantitatively analyze a color change of the substrate on the microfluidic disposable strip.

26. A method of ascertaining a health condition of a user, comprising:
measuring an optical transmittance through a contact lens solution to obtain information about biomarkers associated with the contact lens solution; and
sending information about biomarkers from a contact lens to a computing device.

27. The method of claim 26, wherein the information is obtained at least by measuring a first optical transmittance at a first wavelength through a contact lens solution and a second optical transmittance at a second wavelength through the contact lens solution.

28. The method of claim 27, wherein the first wavelength and the second wavelength are within an infrared range of wavelengths.

29. The method of claim 26, wherein the information is sent from a contact lens container, where the contact lens container comprises:
a light source;
a diffraction grating positioned to diffract light from the light source;
a tilt mechanism attached to the diffraction grating.

30. The method of claim 29, wherein the contact lens container further comprises a slit positioned to receive at least some of the light from the light source after being diffracted with the diffraction grating;
wherein the tilt mechanism is configured to different a range of wavelengths through the slit by positioning the diffraction grating; and wherein the method further comprises:
transmitting a first wavelength of light through a contact lens solution within the contact lens container;
obtaining a first optical transmittance measurement of the first wavelength through the contact lens solution;
transmitting a second wavelength of light through the contact lens solution by moving the diffraction grating with the tilt mechanism; and
obtaining a second optical transmittance measurement of the second wavelength through the contact lens solution.
